SEQUENCE LISTING (13 sequences)

SEQ ID NO: 1 - protein, 6 aa, Artificial Sequence
  sequence promoting adherence of cells
KQAGDV

SEQ ID NO: 2 - protein, 5 aa, Artificial Sequence
  sequence promoting adherence of cells
YIGSR

SEQ ID NO: 3 - protein, 4 aa, Artificial Sequence
  sequence promoting adherence of cells
REDV

SEQ ID NO: 4 - protein, 5 aa, Artificial Sequence
  sequence promoting adherence of cells
IKVAV

SEQ ID NO: 5 - protein, 10 aa, Artificial Sequence
  sequence promoting adherence of cells
RNIAEIIKDI

SEQ ID NO: 6 - protein, 10 aa, Artificial Sequence
  sequence promoting adherence of cells
KHIFSDDSSE

SEQ ID NO: 7 - protein, 5 aa, Artificial Sequence
  sequence promoting adherence of cells
VPGIG

SEQ ID NO: 8 - protein, 7 aa, Artificial Sequence
  sequence promoting adherence of cells
FHRRIKA

SEQ ID NO: 9 - protein, 4 aa, Artificial Sequence
  sequence promoting adherence of cells
KRSR

SEQ ID NO: 10 - protein, 21 aa, Artificial Sequence
  sequence promoting adherence of cells
NSPVNSKIPKACCVPTELSAI

SEQ ID NO: 11 - protein, 4 aa, Artificial Sequence
  sequence promoting adherence of cells
APGL

SEQ ID NO: 12 - protein, 9 aa, Artificial Sequence
  sequence promoting adherence of cells
AAAAAAAAA

SEQ ID NO: 13 - protein, 4 aa, Artificial Sequence
  sequence promoting adherence of cells
RGDS

(19) United States
(12) Patent Application Publication
(10) Pub. No.: US 2019/0201349 A1
(43) Pub. Date: Jul. 4, 2019

(54) CAPSULES AND PARTICLES AND USES THEREOF

(71) Applicant: Yissum Research Development Company of the Hebrew University of Jerusalem Ltd., Jerusalem (IL)

(72) Inventors: Meital RECHES, Bet Hasmonai (IL); Sivan NIR, Tel-Aviv (IL)

(73) Assignee: Yissum Research Development Company of the Hebrew University of Jerusalem Ltd., Jerusalem (IL)

(21) Appl. No.: 15/770,924

(22) PCT Filed: Oct. 26, 2016
(86) PCT No.: PCT/IL2016/051150

§ 371 Date: Apr. 25, 2018

Related U.S. Application Data
(60) Provisional application No. 62/246,437, filed on Oct. 26, 2015.

(51) Int. Cl. A61K 9/48; A61L 31/16; A61L 31/14; A61L 31/08; A61K 33/242; A61K 31/7036; A61K 33/00; A61K 31/704; A61K 9/16; B01J 13/18; C09D 5/00; C09D 189/00

(52) U.S. Cl. CPC A61K 9/4816; A61L 2300/416; A61L 31/146; A61L 31/143; A61L 31/08; A61L 31/088; A61K 33/242; A61K 31/7036; A61K 33/00; A61K 9/4833; A61K 31/704; A61K 9/1694; A61K 9/1617; B01J 13/185; C09D 5/00; C09D 189/00; A61L 2300/606; A61L 2300/404; A61L 31/16

(57) ABSTRACT

Provided are antifouling particles and uses thereof in methods of anti-biofouling.

### TECHNOLOGICAL FIELD

The invention generally concerns novel processes for fabrication particles and capsules of antifouling materials.

### BACKGROUND

Biofouling is a process in which organisms and biomolecules accumulate on a surface, initiated with the adsorption of biomolecules such as proteins and polysaccharides, forming a conditioning film. This film allows the bacteria to attach to surfaces and by that allows the formation of a well-defined bacterial network, termed biofilm. The biofilm produces metabolites that promote the attachment of other organisms. This process eventually results in a thick layer of foulants.

The biofilm, a multicellular community of bacteria, relies on the intercellular exchange of chemical signals called quorum sensing. Owing to this cell-to-cell communication, the behavior of the biofilm can be coordinated; it becomes pathogenic and can resist antibiotic treatment. The formation of biofilm on medical devices and implants may lead to severe infection, which may result in the death of patients.

One proposed solution for biofilm formation on medical devices is to include antibiotics in a releasing system. For example, catheters loaded with a ‘smart’ leaching system, which allows the slow and controlled release of antibiotics to the catheter's surroundings, prevent bacterial accumulation and infection. These systems are not only limited to antibiotics—they can also release metal nanoparticles such as silver and copper. In another approach, biocides are anchored to the device itself. These biocides either eradicate the bacteria that come into contact with the surface or produce chemical species, such as reactive oxygen species (ROS) that abolish the bacteria near the surface. Owing to the increase in bacterial strains that are resistant to antibiotics and biocides, other approaches attempt to use natural compounds such as plants extracts, marine metabolites, and enzymes.

Antifouling approaches and products prevent fouling. They target the first step of biofilm formation—the establishment of the conditioning film. These materials alter the surface energy of the surface (either chemically or physically) so that the adsorption process will be less favorable. One possible approach is to change the topography of a surface. This approach is inspired by natural surfaces such as the skin of a shark or a lotus leaf, which utilize their surface features to prevent fouling. Different coatings can also hinder the interactions of biomolecules and organisms with the surface.

Recently a series of peptides, e.g., tripeptide, have been reported to self-assemble into a coating with antifouling activity [1]. The peptide sequence contains three elements that enable: (i) self-assembly into a film, (ii) adsorption onto any substrate, and (iii) antifouling activity. As the coating procedure is effortless because the peptides form coatings spontaneously by self-assembly on the substrate, and as the assemblies are not limited to a specific substrate and can be applied on metals, oxides, and polymers, the peptides provide a unique and simple approach for preventing biofouling.

### LIST OF REFERENCES

### [1] WO 2014/118779

### GENERAL DESCRIPTION

The inventors of the present invention have developed a family of novel and highly improved material particles, formed by self-assembly of amino-acid based materials, e.g., peptides, upon interaction or contact of the materials with water. The particles so formed have been found to be highly effective biofouling materials, and also effective as carriers of one or more additional functional materials. Thus, the particles of the invention are active carriers of materials suitable for inducing a combination of effects in addition to their antifouling capabilities.

These particles materials are, in part, materials disclosed in non-particulate forms, as antifouling materials, in WO 2014/118779 [1] and US applications derived therefrom, e.g., U.S. application No. 62/215,278, each of which herein incorporated by reference. In some embodiments of the present invention, the particles and capsules of the invention are provided or used in combination with materials in non-particulate form. Thus, where such combinations are disclosed, and unless otherwise and specifically indicated, the materials that are not in particulate form, or which are said to be in non-particulate forms, are materials disclosed in WO 2014/118779 and applications derived therefrom, all incorporated herein by reference.

In a first aspect, there is provided a process for forming particles of a material having at least one surface binding moiety or group, at least one antifouling moiety, and optionally at least one amino acid moiety, the process comprising contacting said material (being in a non-particulate form) with an aqueous medium under conditions permitting transformation (self-assembly) of said material into a particulate form (particles), the particles having porosity that is dependent on the acidity of the aqueous medium.

In some embodiments, upon formation of the particles, the particles may be separated from the aqueous medium.

Upon contact with an aqueous medium, the materials transform or self-assemble or undergo a structural modification into particulate (e.g., particles, capsules) forms. As used herein, the particles are typically of a spherical shape and may be characterized by surface pores forming an internal or surface volumes or cavities (a microcavity) which may or may not contain an amount of a material from which the particle is made or any material or liquid material, e.g., water, present in the aqueous medium in which the particles are made. As such the particles may in fact be regarded as capsules for holding one or more materials.

The particles or capsules of the invention comprise a plurality of pores which define the particles' porosity. As shown, the particles of the invention are of variable porosity. The porosity (namely, inter alia, the density of pores, the average size of the pores) depends, inter alia, on the acidity (pH) of the aqueous medium in which the particles are formed. In some cases, the porosity permits material (solid, liquid, gaseous material, solutes etc) communication between the outside surface of the particles, namely the environment/medium in which the particles are present and their core, i.e. microcavity. In other words, in such cases the degree of porosity permits entrapment of materials in the cavity or pores in the particles via material penetration/absorption/transfer/entrapment through the pores (or via self-assembly in the presence of the material), and thus may be used to selectively entrap material of various or predefined sizes, and similarly control, adjust or arrest release of materials from within the core or internal cavities in the particles.

The porous surface surrounds a plurality of microporous regions being the core or microcavities within the particles. The pore structure, mean flow pore (MFP) size, pore rating and pore diameters may vary from one region of the particle to another, between different types of particles or between populations of particles. As demonstrated herein, however, the porosity of the particles may be adaptable or engineered or pre-selected.

As used herein “adaptable porosity” or generally “particle porosity” refers to the innovative process of the invention that permits manufacture of particles with surfaces of high pore densities as well as particles with surfaces of low pore densities, or generally with a desired pore density, by adapting the conditions under which the particles are formed. As known in the art, a pore density may be determined, for example, by scanning electron microscopy. Based on such and other analyses, the number of pores calculated to be in a given square area can be normalized to a particular reference area.

In general, particles of the invention are characterized by pore densities of between about 10 pores/mm² (10 pores per millimeter square) and about 10 pores/100 μm² (10 pores per micrometer square). In some embodiments, the pore density is between about 10 pores/mm² and about 10 pores/90 μm², between about 10 pores/mm² and about 10 pores/80 μm², between about 10 pores/mm² and about 10 pores/70 μm², between about 10 pores/mm² and about 10 pores/60 μm², between about 10 pores/mm² and about 10 pores/50 μm², between about 10 pores/mm² and about 10 pores/40 μm², between about 10 pores/mm² and about 10 pores/30 μm², between about 10 pores/mm² and about 10 pores/20 μm², between about 10 pores/mm² and about 10 pores/10 μm², between about 10 pores/mm² and about 10 pores/5 μm², between about 10 pores/mm² and about 10 pores/2 μm² or between about 10 pores/mm² and about 10 pores/1 μm².

In some embodiments, the pore density is between about 20 pores/mm² and about 10 pores/100 μm², between about 30 pores/mm² and about 10 pores/100 μm², between about 40 pores/mm² and about 10 pores/100 μm², between about 50 pores/mm² and about 10 pores/100 μm², between about 60 pores/mm² and about 10 pores/100 μm², between about 70 pores/mm² and about 10 pores/100 μm², between about 80 pores/mm² and about 10 pores/100 μm², between about 90 pores/mm² and about 10 pores/100 μm², between about 100 pores/mm² and about 10 pores/100 μm², between about 110 pores/mm² and about 10 pores/100 μm², between about 120 pores/mm² and about 10 pores/100 μm², between about 130 pores/mm² and about 10 pores/100 μm², between about 140 pores/mm² and about 10 pores/100 μm², between about 150 pores/mm² and about 10 pores/100 μm², between about 160 pores/mm² and about 10 pores/100 μm², between about 170 pores/mm² and about 10 pores/100 μm², between about 180 pores/mm² and about 10 pores/100 μm², between about 190 pores/mm² and about 10 pores/100 μm² or between about 200 pores/mm² and about 10 pores/100 μm².

As the density and size of the pores are adaptable and may be varied by modulating the pH of the aqueous medium in which the particles are made, the particles may be prepared in a great variety and for a great variety of uses. The process may be adapted for achieving a particular population of particles or particular use by modifying or adjusting the pH of the aqueous reaction medium.

The pH may be varied to basic pH or acidic pH or to neutral pH, as may be necessary. In some embodiments, the pH of the aqueous medium in which the particles are formed is acidic, i.e. pH less than 7. In some embodiments, the pH is below 6, 5, 4, 3, 2 or below 1.

In some embodiments, the material is contacted with an aqueous medium having a basic pH, i.e. pH greater than 7. In some embodiments, the pH is greater than 8, 9, 10, 11, 12 or 13.

In some embodiments, the pH is between 7 and 10. In other embodiments, the pH is between 7 and 9. In some embodiments, the pH is between 2 and 5. In some embodiments, the pH is between 2 and 4, or between 2 and 3.

In some embodiments, the particles are generally spherically shaped. Where the pH of the aqueous media is acidic, as defined, the particles exhibit a more spherical shaped form, demonstrating limited porosity or pores of limited sizes or pore densities.

In cases where basic pH is used, the particles exhibit a more porous form.

In some embodiments, the average diameter of the pores is in the order of tens of nanometers. In some embodiments, the average diameter is between tens of nanometers to hundreds of nanometers.

In some embodiments, the average diameter of the pores is in the order of tens of micrometers. In some embodiments, the average diameter is between tens of micrometers to hundreds of micrometers.

In some embodiments, the average diameter is at most 200 micrometers, at most 100 micrometers, at most 50 micrometers, or at most 30 micrometer.

In some embodiments, the average diameter is between 2 and 50, between 2 and 40, between 2 and 35, between 2 and 30, between 2 and 25, between 2 and 20, between 2 and 15, between 2 and 10, between 10 and 100, between 10 and 90, between 10 and 80, between 10 and 70, between 10 and 60, between 10 and 50, between 10 and 40, between 10 and 30, or between 10 and 20.

In another aspect, the invention provides particles prepared according to a process of the invention.

In another aspect, the invention provides particles of a material having at least one surface binding moiety or group, at least one antifouling moiety, and optionally at least one amino acid moiety, the particles having adaptable porosity. The particles having the characteristics detailed herein.

The processes and products of the invention may be used to entrap, associate, encage, contain or hold one or more active or non-active materials in cavities or pores present in the particles or to the outer surfaces of the particles. The processes and products similarly permit release of such materials from said particles and delivery thereof for a desired purpose or to a desired target. The active or non-active materials may be entrapped, associated, encaged, contained or held in the particles by any physical or chemical interaction. The particles containing or associated with the one or more active or non-active materials may be prepared by one of two possible methods, as follows:

1. Forming particles according to a process of the invention in the presence of the one or more active or non-active materials, thereby permitting the material to be entrapped or associated in the particles cavity or in the particles material; or

2. Forming particles with a predefined porosity adaptable for interaction, association or otherwise encapsulation of the one or more active or non-active materials, such that the porosity enables containment of the one or more active or non-active materials in the pores or microcavities in the particles or such that the particles material is adapted for interaction with the one or more active or non-active materials.

For example, where particles of the invention are utilized as filtration media for irreversibly holding one or more active or non-active materials, the particles may be prepared under a suitable basic pH to exhibit porosity enabling entrapment of the one or more active or non-active materials in the particles pores or microcavities.

In another example, the particles may be utilized as carriers of one or more active or non-active materials. In such cases, one may choose to manufacture particles having low porosity and high surface area to enable surface association with the one or more active or non-active materials.

As used herein, the one or more “active or non-active material” may be any material which association with the particles of the invention and for uses defined herein are desired. In some embodiments, the active material is a biological material or a material suitable for use in medicine, for example an antifouling material, an antibacterial agent, an antioxidant, an antibiotic, an antibody, an anti-cancer drug, a vaccine element or material, an antineoplastic agent, an antiviral agent, a biological agent, a chemical agent, a fusion agent, a cell regeneration agent, a growth factor agent, a hormonal regulatory agent, a pharmaceutical, and others.

In some embodiments, the at least one active material is an antibiotic. In some embodiments, the antibiotic material is selected from Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefinetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; and combinations thereof.

In some embodiments, the at least one active material is an antioxidant. In some embodiments, the antioxidant is selected from arginine pilolate, ascorbic acid, ascorbyl esters of fatty acids, magnesium ascorbyl phosphate, sodium ascorbyl phosphate, ascorbyl sorbate, bioflavonoids, butylated hydroxy benzoic acids, curcumin, dihydroxy fumaric acid, gallic acid, lipoic acid, lysine, melanin, methionine, superoxide dismutase, tocopherol acetate, tocopherol and tocopherol sorbate.

In some embodiments, the active material is at least one anticancer agent, optionally selected from ifosfamide, cyclophosphamide, dacarbazine, temozolomide, nimustine, busulfan, melphalan, enocitabine, capecitabine, carmofur, gemcitabine, cytarabine, tegafur, tegafur uracil, nelarabine, fluorouracil, fludarabine, pemetrexed, pentostatin, methotrexate, irinotecan, etoposide, sobuzoxane, docetaxel, nogitecan, paclitaxel, vinorelbine, vincristine, vindesine, vinblastine, actinomycin D, aclarubicin, idarubicin, epirubicin, daunorubicin, doxorubicin, pirarubicin, bleomycin, peplomycin, mitomycin C, mitoxantrone, oxaliplatin, carboplatin, cisplatin, nedaplatin, interferon-a, interferon-beta, interferon-gamma, interleukin 2 and ubenimex.

In some embodiments, the active material is in particulate form.

In some embodiments, the active material is at least one nanoparticle or in the form of a nanoparticle. In some embodiments, such nanoparticles may be of any material additive or a metallic nanoparticles. In some embodiments, the metallic nanoparticles are gold nanoparticles, silver nanoparticles or any combination thereof.

In some embodiments, the nanoparticles are non-active.

In some embodiments, the active material is at least one metal, in particulate or non-particulate form, the metal being in ionic or metal form. In some embodiments, the metal is selected from K, Ca, Mg, Mn, Au, Ag, Zn, Li, Na and others, in ionic or metal forms.

In some embodiments, the at least one metallic nanoparticle is gold nanoparticles.

In some embodiments, the active material is at least one material that facilitates quorum-sensing inhibition (QSI) or generally inhibits or arrests quorum sensing.

In some embodiments, the active material is at least one anti-biofilm material; namely a material preventing or causing or brining about destruction of a biofilm.

The non-active material may be selected amongst fillers, stabilizers, colorants, hydrophilic materials, hydrophobic materials, surfactants, and others.

Thus, the invention further provides particles or capsules as defined herein entrapping or associating with at least one active or non-active material. Capsules according to the invention may be used in a variety of applications, including, for example, printing applications, diagnostic applications, as filtering media for a variety of industries (such as medicinal, pharmaceutical, food, agricultural, dye, cosmetic, water, etc), for reducing pollutants, purification of gaseous and liquid medium and for reducing, eliminating or preventing formation of biofilms on surfaces of devices, such as medical devices.

In another aspect, there is provided use of particles or capsules of the invention for preventing antifouling by unicellular organisms and for attracting cells from multicellular organisms. In some embodiments, the particles or capsules are selected to be capable of preventing or arresting adsorption of proteins and/or (poly)saccharides and/or (poly)lipids to a surface.

In another aspect, there is provided use of particles or capsules of the invention in a process for entrapping and/or releasing a material, the process comprising contacting said material with a plurality of particles or capsules or with a precursor of said particles or capsules under conditions suitable for causing entrapping of said material in said particles or capsules prior to or after the particles or capsule have been formed. The process for causing entrapment or association of the particles or capsules with the material may be any one of the processes described herein.

In another aspect, the invention provides a process for delivering or targeting a material to specific target, said process comprising contacting said material with particles or capsules according to the invention or with a precursor of said particles or capsules under conditions suitable for causing entrapping of said material in said particles or capsules prior to or after they have been formed and placing said particles or capsules at the specific place, and delivering or causing delivery of the particles or capsules containing the material to a target, under conditions permitting release of the material at the target.

In another aspect, the invention provides an anti-biofouling composition or coating composition comprising particles or capsules according to the invention.

As stated above, the particles or capsules are made of a material comprising at least one surface binding moiety, at least one antifouling moiety and optionally at least one amino acid moiety. As the particles or capsules exhibit both surface association or binding and antifouling capabilities, it is clear that the materials do not lose their ability to associates with a surface region nor their antifouling capabilities upon transformation to the particulate form. Without wishing to be bound by theory, it is thus believed that the particles or capsules, irrespective of their porosity, are constructed of a plurality of materials, such that on average, a combination of the at least one surface binding moiety, at least one antifouling moiety and at least one amino acid moiety extends outwards from the center of the particles or capsules, such that the particles or capsules exhibit all characteristics associated with the materials in their non-particulate form.

The materials making up particles or capsules of the invention comprise at least one surface binding moiety, at least one antifouling moiety and optionally at least one amino acid moiety may be selected from any such materials, e.g., having antifouling properties. In some embodiments, the compounds are of the general Formula I:

J-L-X-B

wherein
J is a surface binding moiety (or group),
X is an antifouling moiety (or group),
L is a covalent bond or a linker moiety linking J and X,
B may be absent or an amino acid moiety, and
each of “-” represents a bond, e.g., a non-hydrolysable bond.

In some embodiments, where L is present, it is bonded to each of J and X via covalent bonds or non-hydrolysable bonds. In some embodiments, where L is absent, J and X may be bonded to each other via any covalent or non-hydrolysable bond.

These compounds/materials may be used in any of the aspects and embodiments disclosed herein.

In a compound of the general formula J-L-X-B, as defined herein, in some embodiments, the surface binding moiety is selected amongst one or more 3,4-dihydroxy-L-phenylalanin (DOPA), DOPA containing moiety, dopamine and trihydroxyphenylalanine.

Independently of the actual nature of the surface-adsorbing group, namely whether it is DOPA or a DOPA derivative, and whether association occurs via a single atom or a group of atoms or via multiple atoms, the surface-adsorbing moiety (element) is capable of adhering and/or capable of maintaining the surface adherence to any surface material. The surface adherence may be maintained even under non-dry conditions such as under aquatic environment, and also under harsher conditions such as high salt concentrations.

In some embodiments, the moiety comprising DOPA is an organic material selected from amino acids and aliphatic materials. In some embodiments, the organic material is an amino acid. In other embodiments, the material is a peptide.

In some embodiments, the DOPA is linked, associated or bonded to an atom along the linker moiety. In some embodiments, the linker is selected from substituted or unsubstituted carbon chain. In some further embodiments, the linker is composed of two or more amino acids. In some embodiments, the linker comprises between 1 to 40 carbon atoms.

In some embodiments, the linker is of the general structure

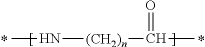

wherein
each * denotes a point of connectivity;
n is between 0 and 40; and
m is between 0 and 40.

In some embodiments, the compound comprises a DOPA unit as well as at least one additional hydroxylated moiety. The hydroxylated moiety may be selected amongst mono-, di-, tri-, tetra- or multiply-hydroxylated alkyls and aryl groups and hydroxylated amino acids.

In some embodiments, the DOPA group or a moiety comprising a DOPA is associated with at least one amino acid.

In some embodiments, the antifouling moiety is a fluorine (—F) atom or a group comprising at least one fluorine atom. In some embodiments, the fluorinated antifouling group is a fluorinated amino acid, as defined.

In a compound of the general formula J-L-X-B, as defined herein, in some embodiments, the antifouling moiety is a fluorinated carbon group. In some embodiments, the fluorinated carbon group is fluorine-substituted carbon group having one or more C—F bonds. In some embodiments, the antifouling moiety comprises 1 or 2 or 3 or 4 or 5 carbon groups, each comprising one or more fluorine atoms. In some embodiments, the fluorinated carbon group comprises or consists —CF, —CF₂, and —CF₃ group(s).

In some embodiments, the fluorinated carbon group is a substituted or unsubstituted alkyl group (carbon group having single C—C bonds), substituted or unsubstituted alkenyl group (carbon group having at least one C═C bond) or substituted or unsubstituted alkynyl group (carbon group having at least one C≡C bond). In some embodiments, the substituted or unsubstituted carbon group, as defined is a C₁-C₂₀ group (being alkyl, alkenyl or alkynyl or mid group equivalents) comprising between 1 and 20 fluorine atoms. Where the group comprises a double bond or a single bond, the minimum number of carbon atoms in the group is 2, and thus the length of the carbon group, or the number of carbon atoms in the group may be selected and adjusted accordingly.

In some embodiments, the substituted or unsubstituted carbon group is selected from a C₁-C₁₉, C₁-C₁₈, C₁-C₁₇, C₁-C₁₆, C₁-C₁₅, C₁-C₁₄, C₁-C₁₃, C₁-C₁₂, C₁-C₁₁, C₁-C₁₀, C₁-C₉, C₁-C₈, C₁-C₇, C₁-C₆, C₁-C₅, C₁-C₄, C₁-C₃, C₁-C₂, C₂-C₂₀, C₂-C₁₉, C₂-C₁₈, C₂-C₁₇, C₂-C₁₆, C₂-C₁₅, C₂-C₁₄, C₂-C₁₃, C₂-C₁₂, C₂-C₁₁, C₂-C₁₀, C₂-C₉, C₂-C₈, C₂-C₇, C₂- C₆, C₂-C₅, C₂-C₄, C₂-C₃, C₃-C₂₀, C₃-C₁₉, C₃-C₁₈, C₃-C₁₇, C₃-C₁₆, C₃-C₁₅, C₃-C₁₄, C₃-C₁₃, C₃-C₁₂, C₃-C₁₁, C₃-C₁₀, C₃-C₉, C₃-C₈, C₃-C₇, C₃-C₆, C₃-C₅, C₃-C₄, C₄-C₂₀, C₄-C₁₉, C₄-C₁₈, C₄-C₁₇, C₄-C₁₆, C₄-C₁₅, C₄-C₁₄, C₄-C₁₃, C₄-C₁₂, C₄-C₁₁, C₄-C₁₀, C₄-C₉, C₄-C₈, C₄-C₇, C₄-C₆, C₄-C₅, C₅-C₂₀, C₅-C₁₉, C₅-C₁₈, C₅-C₁₇, C₅-C₁₆, C₅-C₁₅, C₅-C₁₄, C₅-C₁₃, C₅-C₁₂, C₅- C₁₁, C₅-C₁₀, C₅-C₉, C₅-C₈, C₅-C₇, C₅-C₆, C₆-C₂₀, C₆-C₁₉, C₆-C₁₈, C₆-C₁₇, C₆-C₁₆, C₆- C₁₅, C₆-C₁₄, C₆-C₁₃, C₆-C₁₂, C₆-C₁₁, C₆-C₁₀, C₆-C₉, C₆-C₈, C₆-C₇, C₇-C₂₀, C₇-C₁₉, C₇-C₁₈, C₇-C₁₇, C₇-C₁₆, C₇-C₁₅, C₇-C₁₄, C₇-C₁₃, C₇-C₁₂, C₇-C₁₁, C₇-C₁₀, C₇-C₉, C₇-C₈, C₈-C₂₀, C₈- C₁₉, C₈-C₁₈, C₈-C₁₇, C₈-C₁₆, C₈-C₁₅, C₈-C₁₄, C₈-C₁₃, C₈-C₁₂, C₈-C₁₁, C₈-C₁₀, C₈-C₉, C₉- C₂₀, C₉-C₁₉, C₉-C₁₈, C₉-C₁₇, C₉-C₁₆, C₉-C₁₅, C₉-C₁₄, C₉-C₁₃, C₉-C₁₂, C₉-C₁₁, C₉-C₁₀, C₁₀-C₂₀, C₁₀-C₁₉, C₁₀-C₁₈, C₁₀-C₁₇, C₁₀-C₁₆, C₁₀-C₁₅, C₁₀-C₁₄, C₁₀-C₁₃, C₁₀-C₁₂, C₁₀-C₁₁, C₁₁-C₂₀, C₁₁-C₁₉, C₁₁-C₁₉, C₁₁-C₁₈, C₁₁-C₁₇, C₁₁-C₁₆, C₁₁-C₁₅, C₁₁-C₁₄, C₁₁-C₁₃, C₁₁-C₁₂, C₁₂-C₂₀, C₁₂-C₁₉, C₁₂-C₁₈, C₁₂-C₁₇, C₁₂-C₁₆, C₁₂-C₁₅, C₁₂-C₁₄, C₁₂-C₁₃, C₁₃-C₂₀, C₁₃-C₁₉, C₁₃-C₁₇, C₁₃-C₁₆, C₁₃-C₁₅, C₁₂-C₁₄, C₁₄-C₂₀, C₁₄-C₁₉, C₁₄-C₁₈, C₁₄-C₁₇, C₁₄-C₁₆, C₁₄-C₁₅, C₁₅-C₂₀, C₁₅-C₁₉, C₁₅-C₁₈, C₁₅-C₁₇, C₁₅-C₁₆, C₁₆-C₂₀, C₁₆-C₁₉, C₁₆-C₁₈, C₁₆-C₁₇, C₁₇-C₂₀, C₁₇-C₁₉, C₁₇-C₁₈, C₁₈-C₂₀, C₁₈-C₁₉ and C₁₉-C₂₀.

In some embodiments, the fluorinated carbon group is a C₁-C₂₀ group comprising between 1 and 20 fluorine atoms or a C₂-C₂₀ group comprising between 1 and 20 fluorine atoms.

In some embodiments, the substituted or unsubstituted carbon group comprises 1, 2, 3, 4, 5, 6, 7, 8, 9, 10, 11, 12, 13, 14, 15, 16, 17, 18, 19 or 20 carbon atoms.

In some embodiments, the fluorinated carbon group is perfluorinated (containing only carbon-fluorine bonds (no C—H bonds) and C—C bonds; other heteroatoms may be present). In some embodiments, only the alkyl, alkenyl or alkynyl moieties or groups are perfluorinated, while other moieties or groups present in a compound of the invention are not fluorinated or not perfluorinated.

As disclosed below, the fluorinated group may be an end-group or a mid-group, in which case the alkyl group is an alkylene group, similarly defined as the alkyl group to comprise carbon atoms according to embodiments of the invention, the alkenyl group is an alkenylene, similarly defined, and the alkynyl group is an alkynylene, similarly defined as the alkyl group to comprise carbon atoms according to embodiments of the invention.

In some embodiments, the antifouling moiety is an alkyl. In some embodiments, the alkyl group comprising 1 or 2 or 3 or 4 or 5 or 6 or 7 or 8 or 9 or 10 fluorine atoms. In some embodiments, the antifouling moiety is an alkyl having at least one fluorine atom on each carbon atom.

In other embodiments, the antifouling moiety is a fluorinated substituted or unsubstituted aryl, which may comprise one or more aromatic or heteroaromatic ring systems. The aryl group may be a phenyl group, a substituted phenyl group, a bi- or multi-phenyl system, a fused ring-system (e.g., naphthyl), a multicyclic ring-system or a heteroaryl (which may be a fused or a multicyclic ring-system), each of which being optionally substituted.

In some embodiments, the aryl comprises 1 or 2 or 3 or 4 or 5 fluorine atoms. In some embodiments, where the aryl comprises a single aromatic ring, the ring may be fluorinated by 1 or 2 or 3 or 4 or 5 fluorine atoms. Where the aryl is multicyclic or a fused ring system, such as a naphthyl, each ring in the multicyclic or fused system may comprise at least one fluorine atoms. In some embodiments, the aryl is perfluorinated.

In other embodiments, the aryl is a phenyl group. In other embodiments, the aryl is a fused system, e.g., nathpthyl. In some embodiments, the aryl is a heteroaryl group.

In some embodiments, the phenyl group is substituted or unsubstituted.

In some embodiments, the antifouling moiety comprises or consists one or more fluorinated amino acid moieties.

In some embodiments, the fluorinated amino acid is a fluorinated phenylalanine derivative, wherein the fluorine atom substitutes one or more phenyl ring positions. The substitution on the phenyl ring may be at the ortho, meta and/or para positions. The number of fluoride atoms may be 1, 2, 3, 4, or 5.

Fluorinated amino acids may be an amino acid selected from o-fluorophenylalanine, m-fluorophenylalanine and p-fluorophenylalanine.

The amino acid may be any natural, non-natural in D- or L-configuration or a peptidomemetics. In some embodiments, the amino acid is selected from naturally occurring amino acids and synthetic or semi-synthetic amino acids. In some embodiments, the amino acid is selected amongst alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine valine, pyrrolysine and selenocysteine; and amino acid analogs such as homo-amino acids, N-alkyl amino acids, dehydroamino acids, aromatic amino acids and α,α-disubstituted amino acids, cystine, 5-hydroxylysine, 4-hydroxyproline, α-aminoadipic acid, α-amino-n-butyric acid, 3,4-dihydroxyphenylalanine, homoserine, α-methylserine, ornithine, pipecolic acid, ortho, meta or para-aminobenzoic acid, citrulline, canavanine, norleucine, d-glutamic acid, aminobutyric acid, L-fluorenylalanine, L-3-benzothienylalanine and thyroxine.

In some embodiments, the at least amino acid is an aromatic amino acid. In some embodiments, said aromatic amino acid is selected from tryptophan, tyrosine, naphthylalanine and phenylalanine.

In some embodiments, the amino acid is phenylalanine or a derivative thereof. In some embodiments the phenylalanine derivative is selected from 4-methoxy-phenylalanine, 4-carbamimidoyl-1-phenylalanine, 4-chloro-phenylalanine, 3-cyano-phenylalanine, 4-bromo-phenylalanine, 4-cyano-phenylalanine, 4-hydroxymethyl-phenylalanine, 4-methyl-phenylalanine, 1-naphthyl-alanine, 3-(9-anthryl)-alanine, 3-methyl-phenylalanine, m-amidinophenyl-3-alanine, phenylserine, benzylcysteine, 4,4-biphenylalanine, 2-cyano-phenylalanine, 2,4-dichloro-phenylalanine, 3,4-dichloro-phenylalanine, 2-chloro-penylalanine, 3,4-dihydroxy-phenylalanine, 3,5-dibromo tyrosine, 3,3-diphenylalanine, 3-ethyl-phenylalanine, 3,4-difluoro-phenylalanine, 3-chloro-phenylalanine, 3-chloro-phenylalanine, 2-fluoro-phenylalanine, 3-fluorophenyl alanine, 4-amino-L-phenylalanine, homophenylalanine, 3-(8-hydroxyquinolin-3-yl)-1-alanine, 3-iodo-tyrosine, kynurenine, 3,4-dimethyl-phenylalanine, 2-methylphenyl alanine, m-tyrosine, 2-naphthyl-alanine, 5-hydroxy-1-naphthalene, 6-hydroxy-2-naphthalene, meta-nitro-tyrosine, (beta)-beta-hydroxy-1-tyrosine, (beta)-3-chloro-beta-hydroxy-1-tyrosine, o-tyrosine, 4-benzoyl-phenylalanine, 3-(2-pyridyl)-alanine, 3-(3-pyridyl)-alanine, 3-(4-pyridyl)-alanine, 3-(2-quinolyl)-alanine, 3-(3-quinolyl)-alanine, 3-(4-quinolyl)-alanine, 3-(5-quinolyl)-alanine, 3-(6-quinolyl)-alanine, 3-(2-quinoxalyl)-alanine, styrylalanine, pentafluoro-phenylalanine, phenylalanine, 4-fluorophenyl alanine, 4-iodo-phenylalanine, 4-nitro-phenylalanine, phosphotyrosine, 4-tert-butyl-phenylalanine, 2-(trifluoromethyl)-phenylalanine, 3-(trifluoromethyl)-phenylalanine, 4-(trifluoromethyl)-phenylalanine, 3-amino-L-tyrosine, 3,5-diiodotyrosine, 3-amino-6-hydroxy-tyrosine, tyrosine, 3,5-difluoro-phenylalanine and 3-fluorotyrosine.

In some embodiments, the antifouling moiety comprises between 2 and 12 amino acids, at least one of the amino acids being selected from aromatic amino acids.

In some embodiment, the antifouling moiety comprises one or more fluorine atoms and/or fluorinated moieties.

In some embodiments, the antifouling moiety is a fluorinated organic group.

In some embodiments, the group comprising at least one fluorine atom is F-substituted carbon group having a C—F bond, wherein the number of C—F bonds in the group may be one or more.

In some embodiments, the antifouling moiety comprises 1 or 2 or 3 or 4 or 5 or 6 or 7 or 8 or 9 or 10 fluorine atoms and/or fluorinated moieties.

In some embodiments, the fluorinated carbon group comprises or consists —CF, —CF₂, and —CF₃.

In some embodiments, the fluorinated carbon group is a substituted or unsubstituted alkyl.

In some embodiments, the antifouling moiety is an alkyl comprising 1 or 2 or 3 or 4 or 5 or 6 fluorine atoms.

In some embodiments, the antifouling moiety is an alkyl having at least one fluorine atom on each carbon atom.

In some embodiments, the antifouling moiety is a fluorinated substituted or unsubstituted aryl.

In some embodiments, the aryl comprises 1 or 2 or 3 or 4 or 5 fluorine atoms. In some embodiments, the aryl is perfluorinated. In some embodiments, the aryl is a phenyl group. In some embodiments, the aryl is a heteroaryl group.

In some embodiments, the antifouling moiety comprises or consists one or more fluorinated amino acid moieties. In some embodiments, the fluorinated amino acid is a fluorinated phenylalanine derivative, wherein the fluoride atom substitutes one or more phenyl ring positions. In some embodiments, the fluorinated phenylalanine is selected from o-fluorophenylalanine, m-fluorophenylalanine and p-fluorophenylalanine.

In some embodiments, the amino acid moiety B is absent.

In some embodiments, the amino acid moiety B is present.

Six possible combinations of the three moieties J, X and B are possible. Capsules of the invention may thus be constructed of a material selected from: J-X-B, J-B-X, X-B-J, X-J-B, B-X-J or B-J-X; wherein each “-” designates a covalent bond or a linker moiety.

In some embodiments, the linker moiety is selected from substituted or unsubstituted carbon chains. In some further embodiments, the linker is composed of two or more amino acids. In some embodiments, the linker comprises between 1 to 40 carbon atoms. In some embodiments, the linker moiety is of the general structure:

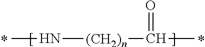

wherein
each * denotes a point of connectivity;
n is an integer between 0 and 40; and
m is an integer between 1 and 40.

In some embodiments, the amino acid moiety B is a single amino acid or a plurality of amino acids forming a peptide moiety.

In some embodiments, the amino acid moiety is at least one amino acid sequence promoting adherence of cells. In some embodiments, the at least one amino acid sequence capable of attracting cells from multicellular organisms.

Thus, particles of the invention may be constructed of materials comprising at least one antifouling moiety, agent (or group), at least one surface-adsorbing moiety (or group), and at least one amino acid sequence promoting adherence of cells, wherein the at least one antifouling moiety is a fluorine (—F) atom or a group comprising at least one fluorine atom; said at least one surface-adsorbing moiety is selected amongst dihydroxy-amino acids and dihydroxy-amino acid containing groups. As such, particles of the invention are antifouling materials capable of preventing or arresting adsorption of organic and/or bio-organic materials (polymers) to a surface (an article's surface) and at the same time capable of promoting and encouraging attachment of cells to the surface. The ability to promote and encourage such attachment of cells is through an amino acid sequence present in a compound of the invention.

The at least one “amino acid sequence promoting adherence of cells” is an amino acid sequence of three or more amino acids, as defined herein, forming as a minimum a tripeptide, and which is selected (inter alia in terms of amino acid identity, connectivity, number of amino acids and sequence length) to provide increased adherence to or adhesiveness of cells. In some embodiments, the amino acid sequence is a truncated fragment of fibronectin which binds integrins (see “Protein-protein Recognition” By Colin Kleanthous). In some embodiments, the truncated fragment is RGD or a fragment comprising RGD.

Examples of at least one amino acid sequence promoting adherence or capable of attracting of cells are RGD; KQAGDV; YIGSR; REDV; IKVAV; RNIAEIIKDI; KHIFSDDSSE; VPGIG; FHRRIKA; KRSR; NSPVNSKIPKACCVPTELSAI; APGL; VRN; and AAAAAAAAA (wherein each letter designates an amino acid as known in the art: A=alanine, C=cysteine, D=aspartic Acid, E=glutamic Acid, F=phenylalanine, G=glycine, H=histidine, I=isoleucine, K=lysine, L=leucine, M=methionine, N=asparagine, P=proline, Q=glutamine, R=arginine, S=serine, T=threonine, V=valine, W=tryptophan and Y=tyrosine).

In some embodiments, the at least one amino acid sequence is RGD (Arg-Gly-Asp).

Thus, in another aspect there is provided a particle comprising at least one surface binding moiety (or group), at least one moiety comprising at least one fluorine atom and an amino acid sequence Arg-Gly-Asp or Arg-Gly-Asp-Ser, wherein each of said moieties being covalently bonded to at least one of the other moieties.

In another aspect, there is provided an antifouling particle comprising the amino acid sequence Arg-Gly-Asp or Arg-Gly-Asp-Ser.

In some embodiments, the antifouling particle comprises at least one fluorinated moiety, as defined.

In some embodiments, the material is a peptide that comprises between 2 and 12 amino acids, each amino acid being selected from aromatic amino acids. In some embodiments, the peptide comprises DOPA at one termini and a fluorinated aromatic amino acid selected from o-fluorophenylalanine, m-fluorophenylalanine and p-fluorophenylalanine at the other termini.

In some embodiments, the peptide comprises DOPA at a mid-point along the peptide and a fluorinated aromatic amino acid selected from o-fluorophenylalanine, m-fluorophenylalanine and p-fluorophenylalanine at each of the peptide termini

In some embodiments, the material making up particles of the invention, each of J, X and B, independently, may appear once or more times; where at least one of J, X and B, independently, is present multiple times, it need not appear in sequence. Thus, multiple J, X and/or B, independently, need not be grouped. Examples of such particles of the invention include particles of the general formulae J-J-X-B-J, J-X-B-X-X, X-B-J-J-B-X, J-X-X-B, J-J-X-X-B, J-J-J-X-X-X-B, X-X-X-J-J-X-X-X-B and others, wherein each of J, X and B is as defined, and wherein the repeating moieties may or may not be the same. For example, in a particle made up of a material having the general structure J-X-X-B, each of the two X moieties may or may not be the same.

In some embodiments, the material making up the particles is a tripeptide, a tetrapeptide, a pentapeptide or a hexapeptide.

In some embodiments, the particle is of a material selected from:

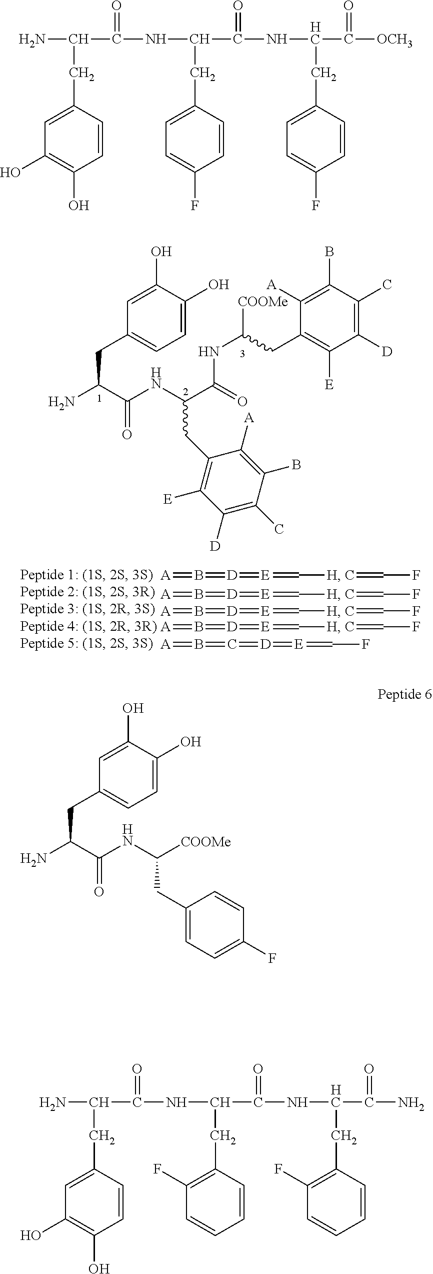

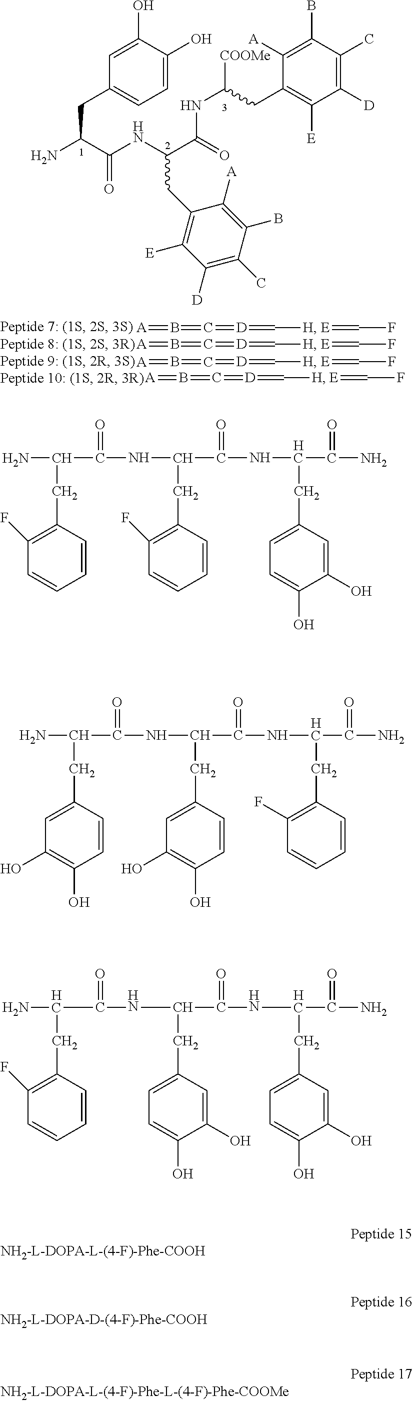

In some embodiments, the material is DOPA-Phe(4F)-Phe(4F)-Arg-Gly-Asp, herein designated Peptide 18.

In some embodiments, the compound is a peptide herein designated Peptide 19, having the full structure:

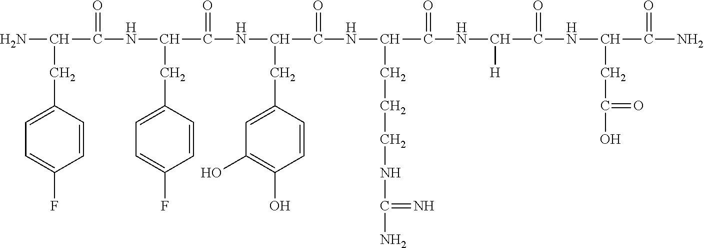 [SMILES: N=C(N)NCCCC(NC(=O)C(Cc1ccc(O)c(O)c1)NC(=O)C(Cc1ccc(F)cc1)NC(=O)C(N)Cc1ccc(F)cc1)C(=O)NCC(=O)NC(CC(=O)O)C(N)=O]

In accordance with the invention, the particles are selected to self-assemble into films. Thus, when provided on a surface region of a substrate, the material associate with the surface region via at least one surface-adsorbing moiety. The surface binding moiety is one having at least one atom or group of atoms capable of interacting, e.g., by adsorption, to at least one surface region. In some embodiments, the at least one surface binding moiety is selected amongst 3,4-dihydroxy-L-phenylalanin (DOPA), a DOPA containing moiety and dopamine

The at least one surface binding moiety, e.g., DOPA or a DOPA containing moiety, is configured to adhere, adsorb or associate with a surface or a region of a surface which protection against fouling is desired. The term “associate” or “adhere” or “adsorb”, as used herein, refers to any physical or chemical interaction to be formed between the surface and the DOPA group or any atom thereof. The association may be via Van-der-Walls, coordinative, covalent, ionic, electrostatic, dipole-dipole, or hydrogen association (bond or interaction).

In another aspect, the invention provides use of a particle according to the invention for adsorbing materials, e.g., contaminates, from a surrounding medium. In some embodiments, the particles are associated to a surface that is in direct contact with a medium (gaseous or fluid) which contains, is expected to contain, or is susceptible to contain an amount of a material which removal from the medium is desired. When particles of the invention come into contact with said material, the material is absorbed in the particle (capsule), in any one or more of the particle cavities or pores, or on its surface.

In some embodiments, the material to be absorbed or contained or removed from the medium by particles of the invention, may be a contaminating material, an impurity, a material of a specific size or composition which association with the particle may be by adsorption, absorption, hydrogen bonding, electronic association etc, or any other material. As the particles exhibit antifouling characteristic, they provide the dual ability of antifouling and filtration.

In some embodiments, the medium may be also enriched with the antifouling materials in non-particulate form. Thus, in such embodiments, one or both of the particulate materials and the non-particulate materials may be associated with a surface and the other maintained free in the medium (gaseous or fluid, liquid).

In a further aspect, the invention provides a medical device or implant having at least a surface region thereof coated with a plurality of particles according to the invention.

In a further aspect, the invention provides a method for reducing fouling on a surface, the method comprising the step of applying a plurality of particles according to the invention to a region of the surface.

In some embodiments, the particles are suitable for preventing or arresting or minimizing or diminishing one or more of the following:

(a) adsorption of organic and/or bio-organic materials to a surface;

(b) adsorption of proteins and/or (poly)saccharides and (poly)lipids to a surface;

(c) secretion from cells of multi-organism or of micro-organisms onto a surface; and

(d) adsorption of cells of multi-organism or micro-organisms to a surface.

The invention further provides a formulation comprising a plurality of particles according to the invention.

In some embodiments, the formulation comprises a population of particles, at least 50% of which being particles or capsules according to the invention.

In some embodiments, the formulation comprises at least 50% particles or capsules according to the invention and at least an amount of the materials in non-capsule form. In some embodiments, where the formulation comprises an amount of the material from which the particles or capsules are formed (present in a non-particulate form), the formulation may comprise at least 50%, 55%, 60%, 65%, 70%, 75%, 80%, 85%, 90%, 95%, 96%, 97%, 98%, 99% of particles or capsules according to the invention. Thus, the invention further contemplates formulations comprising materials as described herein in non-particulate and particulate forms.

In some embodiments, the formulations are adapted for forming a film of a compound or combination of compounds/peptides, as defined, on a surface region. In some embodiments, the formulation comprises at least one volatile liquid carrier which carries particles or capsules or particles combination in solution, as a suspension or as dispersion.

In some embodiments, the formulation is configured to provide a film capable of promoting attachment of cells thereonto and/or capable of reducing biofouling.

In some embodiments, the formulation is utilized in a method of implantation (e.g., establishing an implant in vivo, in situ). In some embodiments, the formulation is configured to provide a film on a surface region of an implantable device or object.

In other embodiments, the formulation is utilized in a biotechnological process involving adherence of cells, e.g., eukaryotic cells, to a surface, while preventing biofouling. In some embodiments, the surface may be a surface region of a biotechnological device such as a reactor used in biotechnology.

In some embodiments, the film is adapted for preventing contamination by an organism at the implantation site. In some embodiments, the film is configured for preventing biofouling caused by an organism.

In some embodiments, the microorganism is selected from bacteria, diatoms, hydroids, algae, bryozoans, protozoans and ascidians.

In some embodiments, the organism is bacteria. In some embodiments, the bacteria is selected from Bordetella pertussis, Borrelia burgdorferi, Brucella abortus, Brucella canis, Brucella melitensis, Brucella suis, Campylobacter jejuni, Chlamydia pneumonia, Chlamydia psittaci, Chlamydia trachomatis, Clostridium botulinum, Clostridium difficile, Clostridium perfringens, Clostridium tetani, Corynebacterium diphtheria, Enterococcus faecalis, Enterococcus faecium, Escherichia coli (E. coli), Enterotoxigenic Escherichia coli (ETEC), Enteropathogenic E. coli, Francisella tularensis, Haemophilus influenza, Helicobacter pylori, Legionella pneumophila, Leptospira interrogans, Listeria monocytogenes, Mycobacterium leprae, Mycobacterium tuberculosis, Mycoplasma pneumonia, Neisseria gonorrhoeae, Neisseria meningitidis, Pseudomonas aeruginosa, Rickettsia rickettsii, Salmonella typhi, Salmonella typhimurium, Shigella sonnei, Staphylococcus epidermidis, Staphylococcus saprophyticus, Streptococcus agalactiae, Streptococcus mutans Streptococcus pneumonia, Streptococcus pyogenes, Treponema pallidum, Vibrio cholera, Vibrio harveyi and Yersinia pestis.

In some embodiments, the bacterium is Escherichia coli (E. Coli). In some other embodiments, the bacterium is P. aeruginosa.

In some embodiments, the film is adapted for inducing attachment of cells onto a surface region of the implantable device or object.

As used herein, the term “implant” refers to any medical device, e.g., medical devices that are adapted for insertion into a body cavity. An implant of the invention is manufactured to be inserted in the body in order to facilitate proper bodily function or treat at least one clinical condition. Some implants replace a missing biological structure, support a damaged biological structure, or enhance an existing biological structure. In some other embodiments, an implant can provide a function clinically needed by the subject. The surface of the implant coming into contact with a body region or organ or tissue may be of a biomedical material such as titanium, silicone or apatite. The implant may be dental, orthodental, orthopedic etc.

In some embodiments, the implant is a bone implant and the film formed thereon is suitable for anchoring osteoclasts or osteoblasts on the implant surface.

Thus, in another aspect, the invention provides a method of anchoring or attracting cells onto a surface region of an implantable device or object, said method comprising forming on a surface region of the implantable device or object a film of particles according to the invention and implanting said device or object, wherein the film promotes anchoring or attachment of cells thereto.

As used herein, the term “cells” refers to cells such as to osteoblasts or osteoclasts.

In another aspect, the invention provides a method of reducing biofouling caused by an organism on an implant post-operative placement of said implant in a body cavity or in contact with a tissue, said method comprising forming on a surface region of the implant a film of particles according to the invention and implanting said implant, wherein the film promotes anchoring or attachment of cells thereto.

In another aspect, the invention provides a method of attracting body cells to and reducing biofouling on an implant, said method comprising forming a film of particles according to the invention on a surface region of the implant and implanting said implant.

In another aspect, the invention provides a method for preventing, ameliorating, or treating a disease or disorder related to an implant having been placed in a body cavity or on a tissue, said method comprising the step of forming a film of particles according to the invention on a surface region of an implant prior to implantation.

In some embodiments, the disease is peri-implantitis.

In another aspect, the invention provides a method for prolonging lifetime of an implant intended for implantation in a subject, said method comprising coating a surface region of the implant prior to implantation with a film comprising particles according to the invention.

In another aspect, the invention provides a method for stimulating or encouraging bone healing or absorption of an implant after implantation, the method comprising coating a surface region of the implant prior to implantation with particles of the invention, thereby inducing bone healing.

In another aspect, the invention provides a medical device comprising a film of particles according to the invention. In some embodiments, the implant is a dental implant. In some embodiments, the implant is a permanent implant.

In another aspect, the invention provides a kit comprising a formulation or suspension of particles according to the invention and instructions of use.

As used herein, the subject may be a human or a non-human subject.

In some embodiments, the surface region of an implant is any one region of an implant surface, being optionally the full surface of the implant, as determined by a medical professional or one versed in the pertinent field.

As may be realized from the disclosure provided herein, the self-assembly or transformation of the materials into particle forms may be used to initiate, hasten or otherwise force rearrangement of non-particulate form materials into particles. Thus, in a process and product according to the invention, based on materials of the general Formula I, the materials may rearrange to form the pores or cavities, may be embedded in a pores or cavities, etc.

Thus, in another aspect, the invention provides a process for preparing a particle form of at least one material, the process comprising contacting said at least one material in the presence of a compound of general formula J-L-X-B, in an aqueous medium, under conditions permitting self-assembly of said at least one material into a particle form, wherein each of J, L, X, B and “-” are as defined herein.

In some embodiments, said the compound of the formula J-L-X-B is entrapped within pores or cavities, on the surface of the particles and/or within the microcavities of the particles. Thus, in some embodiments, where the process of the invention is not intended for manufacturing particles of other materials, the particles of the invention consist of a material selected amongst compounds of the formula J-L-X-B. Where the process of the invention is intended and adapted for forming particles of another material, the particles of the invention may comprise a material selected amongst compound of formula J-L-X-B.

As may be understood from the disclosure herein, once obtained, the particles or capsules of the invention may be modified or otherwise treated to modify their properties. This may be achieved, inter alia, by modifying the particles outer surface, e.g., by associating the surface with ligand molecules or other agents, in a reversible or non-reversible fashion, to thereby achieve one or more of the following:
1. improve or otherwise modify or modulate solubility;
2. improve or otherwise modify or modulate stability;
3. modulate the particles hydrophobicity/hydrophilicity;
4. allow association of active and non-active materials, as defined herein, to the surface of the particles, rather than or in combination with encapsulation of such materials in the particles cavities or pores;
5. modulate slow, controlled, sustained or rapid release of active or non-active agents contained within or on the surface of the particles or capsules;
6. induce or prevent particle aggregation;
7. link or associate the particles or capsules of the invention to linker moieties, matrix material, polymers, and other solid or liquid materials;
8. use the particles or capsules as drug delivery systems for medicinal use.

In some embodiments, the particles of the invention are associated with at least one linker moiety, as defined herein, after its production according to methods of the invention. In some embodiments, the linker moieties may be activated for further association with an active or non-active material.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-C show Scanning Electron Microscopy (SEM) pictures of self-assembled structures of a peptide of the invention: FIG. 1A—in tris buffer, at pH 8.5;

FIG. 1B—in HCl 1M; and FIG. 1C—polymerized particles.

FIGS. 2A-C provide optical microscope images of free fluorescent doxorubicin and encapsulated doxorubicin in particles of a peptide of formula I: FIG. 2A-no peptide;

FIG. 2B—in tris buffer, pH8.5; and FIG. 2C in HCl 1M.

FIGS. 3A-B provide Transmission Electron Microscope (TEM) of a peptide of Formula I, which is self-assembled in water in the presence of gold nanoparticles.

FIG. 4 demonstrates an antifouling assessment by protein adsorption.

FIGS. 5A-G provide the following: FIGS. 5A-B show Scanning Electron Micrographs of (FIG. 5A) symmetric spheres formed under acidic conditions, 1M HCl and (FIG. 5B) ‘spikey spheres’ formed under basic conditions, 10 mM tris buffer, pH=8.5. FIGS. 5C-E show contact angles of (FIG. 5C) a bare glass slide, (FIG. 5D) a glass slide coated with the symmetric spheres, and (FIG. 5E) a glass slide coated with the ‘spikey spheres’. FIGS. 5F-G show FT-IR spectra of the peptide assemblies: (FIG. 5F) Symmetric spheres, and (FIG. 5G) ‘spikey spheres’.

FIGS. 6A-B provide assessment of the antifouling activity of the modified surfaces. (FIG. 6A) The graph plots the adsorbed amounts of BSA on the modified surfaces. (FIG. 6B) The plot shows the amount of bacteria on the modified surfaces. Error bars represent standard deviations (n=9).

FIGS. 7A-G provide the following: FIG. 7A-C show fluorescence microscopy micrographs of (FIG. 7A) doxorubicin on a bare substrate, (FIG. 7B) spheres self-assembled under an acidic condition in the presence of doxorubicin, and (FIG. 7C) spheres self-assembled under a basic condition in the presence of doxorubicin. FIGS. 7D-G show doxorubicin release from the peptide assemblies: fluorescence spectrum of doxorubicin released from (FIG. 7D) symmetric spheres and (FIG. 7E) ‘spikey spheres’. Fluorescence intensity at 590 nm as a function of time for doxorubicin released from (FIG. 7F) symmetric spheres and (FIG. 7G) ‘spikey spheres’.

FIG. 8 plots the number of CFUs resulting from surfaces modified with either the peptide assemblies, GOx, or a combination of the assemblies and the GOx. Error bars represent standard deviations (n=24).

FIGS. 9A-H show self-assembly of the peptide in different Tris concentrations. FIGS. 9A-D show the coverage of the surface by the peptide structures, and FIGS. 9E-H show the morphology of the structures. FIGS. 9A and 9B—1 mM, FIGS. 9B and 9F—5 mM, FIGS. 9C and 9D—15 mM, and FIGS. 9D and 9H—20 mM.

FIGS. 10A-F show self-assembly of the peptide at different pH values: FIG. 10A pH=1, FIG. 10B pH=2, FIG. 10C pH=3, FIG. 10D pH=4, FIG. 10E pH=5, and FIG. 10F pH=6. The average diameter of the spheres formed in acidic medium decreases with increasing pH until they merge to produce a film.

FIGS. 11A-H show self-assembly of the peptide at different pH values: FIG. 10A pH=6.5, FIG. 10B pH=7, FIG. 10C pH=7.5, FIG. 10D pH=8, FIG. 10E pH=9, FIG. 10F pH=9.5, FIG. 10G pH=10, and FIG. 10H pH=10.5. At a neutral pH the surface is mostly covered with undefined aggregates. When the pH is increased, ‘spikey spheres’ are formed. The higher the pH, the denser each sphere becomes.

FIG. 12 shows the reduction in the fluorescence intensity of Doxorubicin upon the self-assembly of the peptide.

FIGS. 13A-C show Doxorubicin encapsulation/adsorption. FIG. 13A Doxorubicin in Tris buffer, FIG. 13B the solution becomes opaque with peptide addition, and FIG. 13C stained peptide assemblies precipitate during the night, leaving a clear solution above them.

FIGS. 14A-D show Gentamicin release from the peptide assemblies after overnight incubation. FIGS. 14A and 14B, agar plates on which buffer incubated with bare surfaces was taken. FIG. 14C, zones of inhibition formed after applying buffer that was incubated with surfaces modified with ‘spikey spheres’. FIG. 14D, zones of inhibition formed after applying buffer that was incubated with surfaces modified with symmetric spheres.

### DETAILED DESCRIPTION OF EMBODIMENTS

Various peptides of the invention have been formed into particulate form and have been used in a plurality of applications, such as, inter alia, biofouling, encapsulation of medicinal materials, encapsulation of nanoparticles, etc. The results presented herein demonstrate such uses and the particles broader utility as carriers or materials from which the material may be released or prevented from being released.

### Example 1: Preparation of Particles of the Invention

Preparation of porous particles: A fresh stock solution was prepared by dissolving the peptide in pure ethanol to a concentration of 100 mg/mL. The peptide stock solution was then diluted to a final concentration of 1 mg/mL in 10 mM tris buffer.

Preparation of symmetrical spheres: A fresh stock solution was prepared by dissolving the peptide in pure ethanol to a concentration of 100 mg/mL. The peptide stock solution was then diluted to a final concentration of 1 mg/mL in 1M hydrochloric acid (HCl).

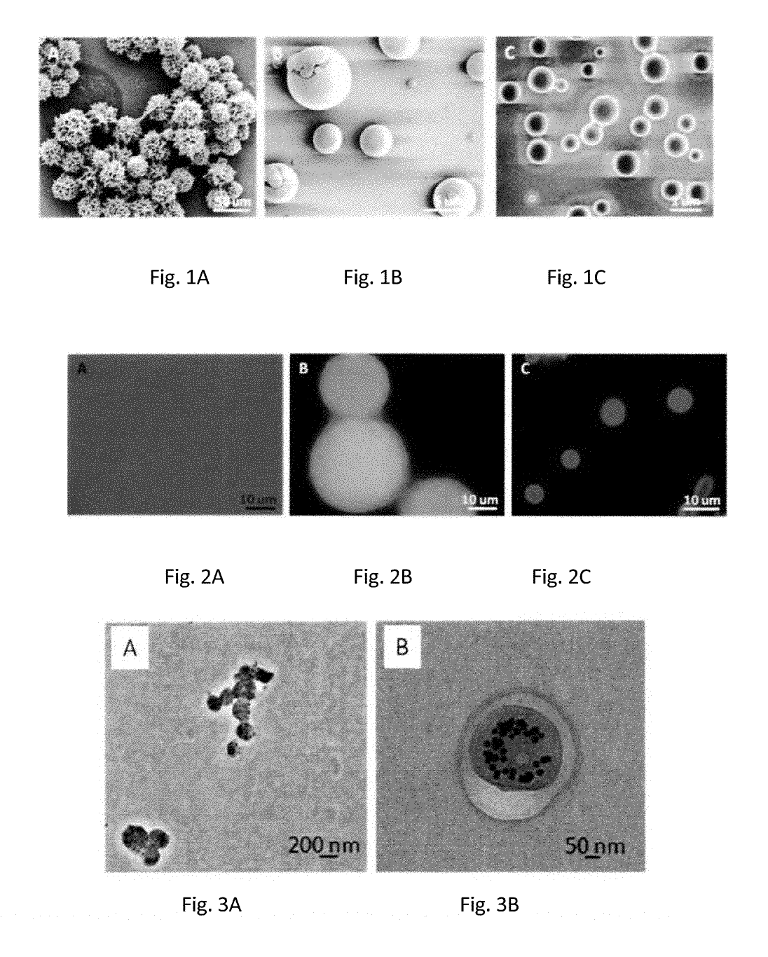

As FIGS. 1A, 1B and 1C provide, the peptide of the invention can self-assemble in an aqueous medium into either spherical or porous particles depending on the pH of the solution with a diameter of tens of micrometers. The morphology of the structures formed in solution of basic pH (pH=8.5) was highly porous (FIG. 1A). Conversely, in acidic pH (1M HCl) the peptide self-assembled into symmetrical spheres (FIG. 1B).

In addition, spherical particles were formed also by polymerization (FIG. 1C).

### Example 2: Encapsulation of Doxorubicin in the Self-Assembled Structures of Peptide

Encapsulation of an active material, e.g., doxorubicin, in a particle of the invention has also been achieved.

Doxorubicin loaded particles were prepared by dilution of a peptide stock solution (100 mg/mL) to a concentration of 1 mg/mL in aqueous medium (either tris buffer or HCl) containing doxorubicin at a concentration of 0.05 mg/mL.

FIG. 2A shows Optical Fluorescent Microscope image of free doxorubicin. Fluorescent microscope image of the peptide structures self-assembled in the presence of doxorubicin, at high pH, is shown in FIG. 2B. Fluorescent microscope image of the peptide structure self-assembled in the presence of doxorubicin, at low pH (1M HCl), is shown in FIG. 2C.

### Example 3: Encapsulation of Gold Nanoparticles in the Self-Assembled Structures of Peptide

Further prepared were particles of a material of formula I in the present of gold particles in the aqueous medium.

Gold nanoparticles loaded particles were prepared by dilution of a peptide stock solution (100 mg/mL) to a concentration of 1 mg/mL in aqueous medium (either tris buffer or HCl) containing gold nanoparticles at a concentration of 100 ug/mL.

Pictures of Transmission Electron Microscope (TEM) show that the peptide having Formula I self-assembled in water in the presence of gold nanoparticles, at a concentration of 2 mg/mL (FIG. 3A) and at a concentration of 1 mg/mL (FIG. 3B).

### Example 5: The Particles Resists Fouling

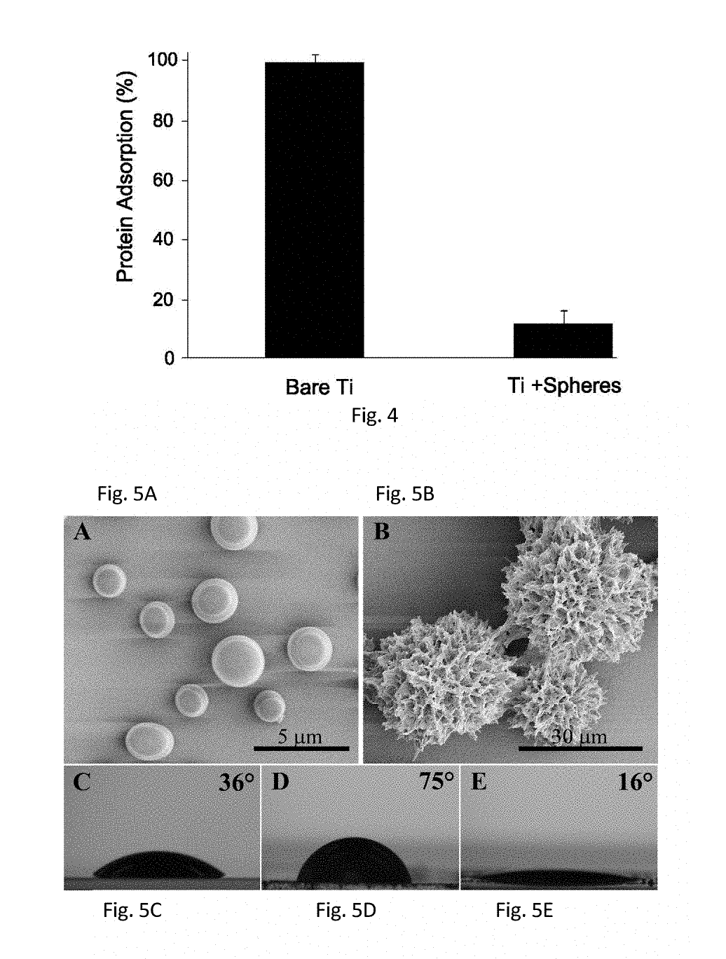

FIG. 4 demonstrates normalized adsorption of Bovine Serum Albumin (BSA) onto titanium substrate coated with the peptide particle, compared to a bare titanium substrate. A reduction in protein adsorption onto titanium surface due to the presence of the spheres particle can be clearly seen.

### Example 6: Self-Assembly of the Tripeptide into Spherical Particles in Either Acidic or Basic Aqueous Solutions

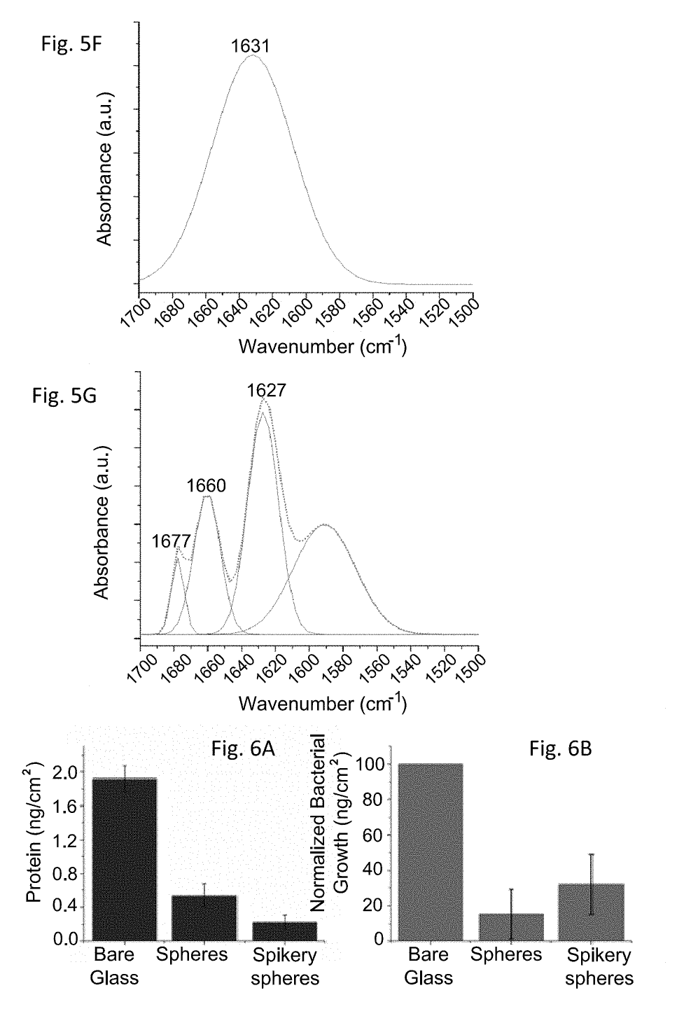

To initiate the self-assembly of the tripeptide, the peptide was dissolved in either an acidic (1M HCl) or basic (Tris buffer, 10 mM, pH=8.5) solution. To characterize the structures formed by the tripeptide under different conditions, Scanning Electron Microscopy (SEM) analysis was obtained. The results revealed the formation of different structures in acidic or basic solutions. In acidic medium, the peptide self-assembles into symmetric spheres with an average diameter of 2.0±0.4 μm. In contrast, in basic medium, the peptide self-assembles into porous spheres with an average diameter of 26±3 μm. These assemblies are termed herein ‘spikey spheres’ (FIGS. 5A-B).

By applying these structures onto a bare substrate the chemical and physical characteristics of the substrate's surface can be altered. The chemical characteristics of the surface will change due to the chemical nature of the peptide (hydrophobic and fluorinated), and the physical properties will change due to the new microtopography gained by these structures. Both features play a major role in the design of efficient antifouling materials.

To coat a surface with these peptide assemblies, the peptides were self-assembled and drop-casted the peptide solution on clean glass slides. After the samples dried in air, the samples were dipped in Triple Distilled Water (TDW) to remove excess and non-adhered peptide from the surface. Finally, they were dried under nitrogen. To determine whether the peptide structures indeed alter the features of the surface, the contact angle of the coated surfaces were measures. Whereas substrates covered with the symmetric spheres exhibited an increase in the contact angle, from 36° for bare glass, to 75° for coated glass, the substrates coated with the ‘spikey spheres’ exhibited a decrease in the contact angle to 16° (FIGS. 5C-E). The increase in the contact angle in the case of the symmetric spheres indicates an increase in hydrophobicity. This could be attributed to the topography and to the hydrophobic features of the peptide.

In the case of the ‘spikey spheres’, the surface became more hydrophilic. This can be explained by the porosity of the structures. In fact, while measuring the contact angle, the inventors could detect that the water droplet was almost completely soaked by the surface.

To obtain information on the secondary structures of the peptide assemblies, Fourier Transform Infrared Spectroscopy analysis (FT-IR) have been performed. The symmetric spheres formed in HCl had a single peak at 1631 cm⁻¹, which corresponds to a β-sheet structure. The ‘spikey spheres’ had a different spectrum, consisting of three distinctive peaks at 1627 cm⁻¹, 1660 cm⁻¹, and 1677 cm⁻¹. These peaks may suggest a more complex structure that may combine both α-helical and β-sheet secondary structures. The different spectra are correlated with the different microstructures formed by the peptide.

To better understand how the ionic strength of the solution affects the self-assembly process, we self-assembled the peptide in different Tris concentrations, ranging from 1 mM to 20 mM and analyzed the morphology of the structures using SEM (FIG. 9) The results indicated that at a concentration of 1 mM, only a few spherical structures formed. However, their appearance was different from the structures obtained in 10 mM Tris. In addition, the majority of the surface was covered with amorphous aggregates. Upon increasing the concentration to 5 mM, ‘spikey spheres’ were formed, but their dispersion on the surface was different. Whereas the spikey spheres formed in 10 mM Tris tend to appear in large clusters, the spheres formed in 5 mM Tris appeared in small clusters of only a few assemblies. Moreover, amorphous aggregates could be detected between the ordered structures, whereas the in-between spacing in the case of 10 mM Tris was free from aggregates. In higher concentrations, such as 15 mM and 20 mM, the amorphous aggregates were dominant. In addition, very few spheres formed, but they were denser and larger than with 10 mM Tris. Accordingly, we decided that the optimal buffer concentration was 10 mM Tris.

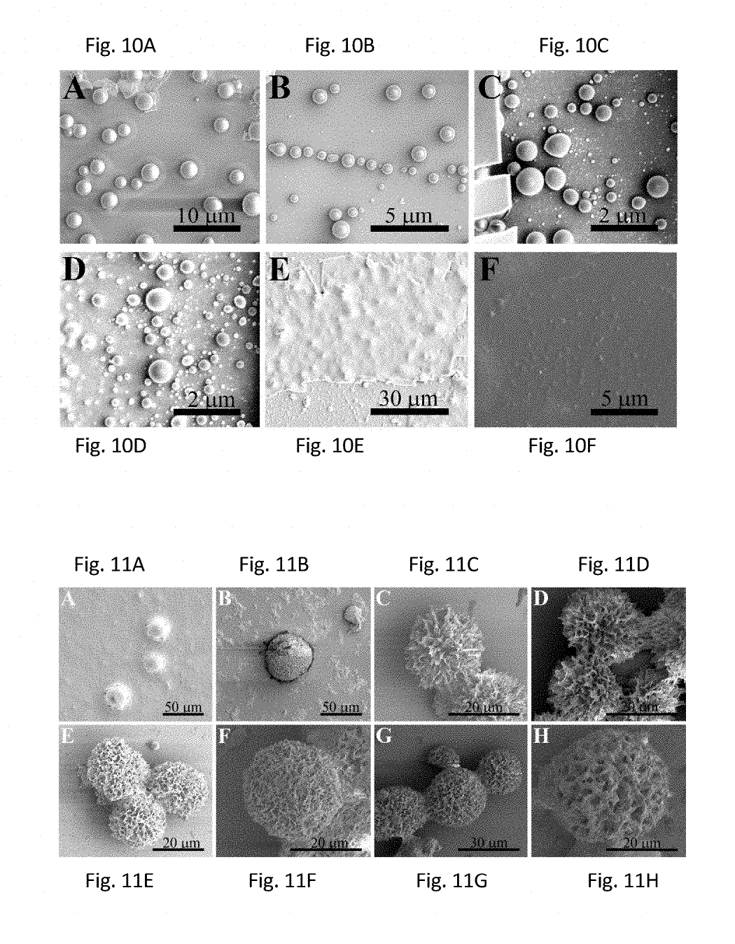

To better understand how the pH value of the solution affects the self-assembly process, self-assembled peptides were achieved in solutions of different pH values, ranging from 1 to 10.5. Since the acidic solutions differ from the basic solution in their chemical composition, they cannot be compared. Interesting trends, however, could be observed in the two different pH series. SEM analysis revealed that lowering the acidity of the solution results in smaller spheres. FIG. 10 presents the alteration of spheres while the pH is increased from 1 to 6. The spheres became increasingly smaller, until no spheres could be detected, and a uniform coating was formed. With the basic medium, upon an increase in the pH value, the ‘spikey spheres’ became denser and less spikey (FIG. 11). According to these results, we can conclude that by fine-tuning of the pH values, we can control the physical properties of the spheres and their appearance.

To determine the antifouling activity of surfaces decorated with these assemblies, we first investigated their resistance to protein adsorption. Bare glass slides and peptide-coated glass slides were incubated in a solution of Bovine Serum Albumin (BSA) at a concentration of 150 μM, for two hours at 37° C. To determine the adsorbed amounts of BSA on the substrate, the Non-interfering protein Assay™ kit was used. The plot in FIG. 6A summarizes the results. The amount of protein adsorbed on the bare glass substrates was substantially higher than the amount of protein adsorbed on the peptid-coated glass substrates. The symmetric spheres, formed in acidic medium, reduced the amount of protein from 1.9 nmol/cm² to 0.5 nmol/cm². The ‘spikey spheres’ formed in basic medium were found to be even more efficient, and reduced the amount of adsorbed protein, from 1.9 nmol/cm² to 0.28 nmol/cm². Previous reports showing the higher tendency of BSA to adsorb onto hydrophobic surfaces compared with hydrophilic surfaces support these results. It was also found that BSA undergoes denaturation and spatial rearrangements while adsorbing to surfaces. Its spatial orientation depends on the surface vacancy, which allows it to spread. Since the ‘spikey spheres’ form non-smooth surfaces with complex topography, they may be more effective against the protein, compared with the spherical spheres.

To assess the extent of the bacterial attachment to the surface, bare and peptide-modified surfaces were incubated with Escherichia coli overnight to allow the adsorption of bacteria and the formation of biofilms on the substrates. After incubation, the bacteria were removed from the substrates by sonication, diluted 10-fold, plated, and the colonies forming units (CFUs) were counted. The surfaces coated with the different peptide assemblies exhibited antifouling activity, and the number of colonies counted from these samples was lower than the number of colonies from the bare substrates. The ‘spikey spheres’ reduced the bacterial growth by 68%, but importantly, the spherical spheres reduced it by 85% (FIG. 6B). Similar results were also obtained for longer incubation times.

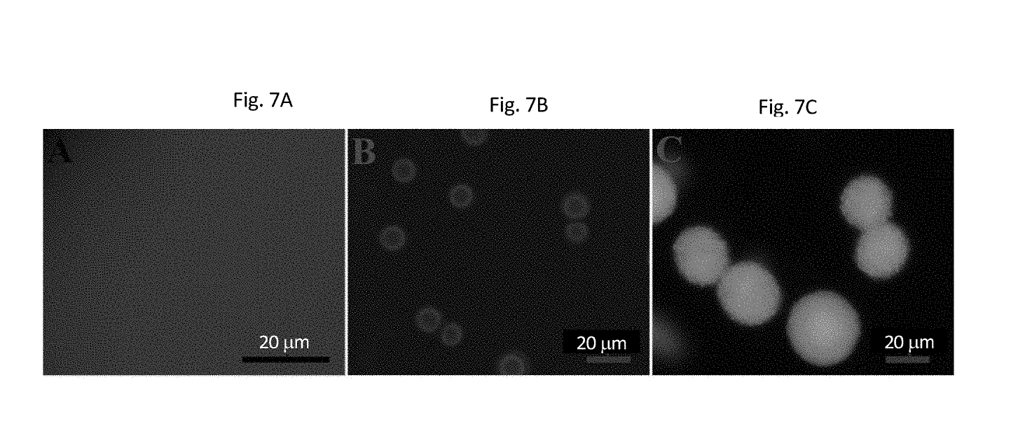

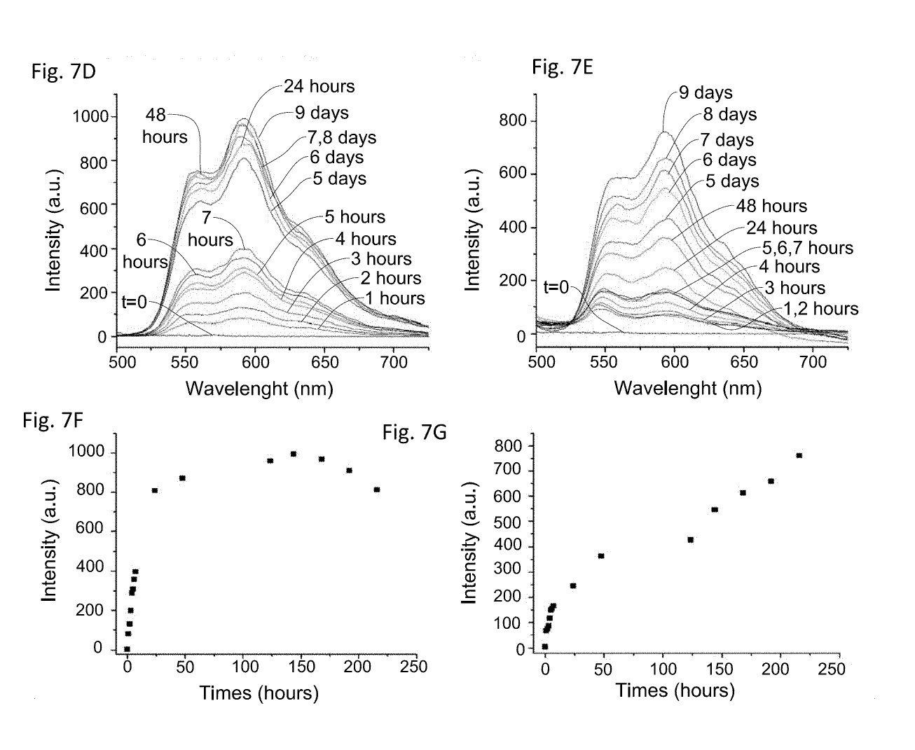

To improve the antifouling activity of the peptide assemblies, we decided to exploit their ability to adsorb or encapsulate and then release active compounds. To determine this capability, we self-assembled the peptide in the presence of 50 μg/mL of the fluorophore molecule, doxorubicin. After a few minutes of incubating them together, we drop-casted the solution onto a glass cover slip and dried it under ambient conditions. The glass was then rinsed with water to remove the excess dye and non-adsorbed peptide. Fluorescence microscopy analysis revealed that glass slides casted with only doxorubicin exhibited a fully stained surface, whereas the slides casted with the peptide and dye exhibited clearly distinct fluorescent spheres. The drug specifically adsorbed onto the peptide assemblies, without affecting their structure (FIG. 7).

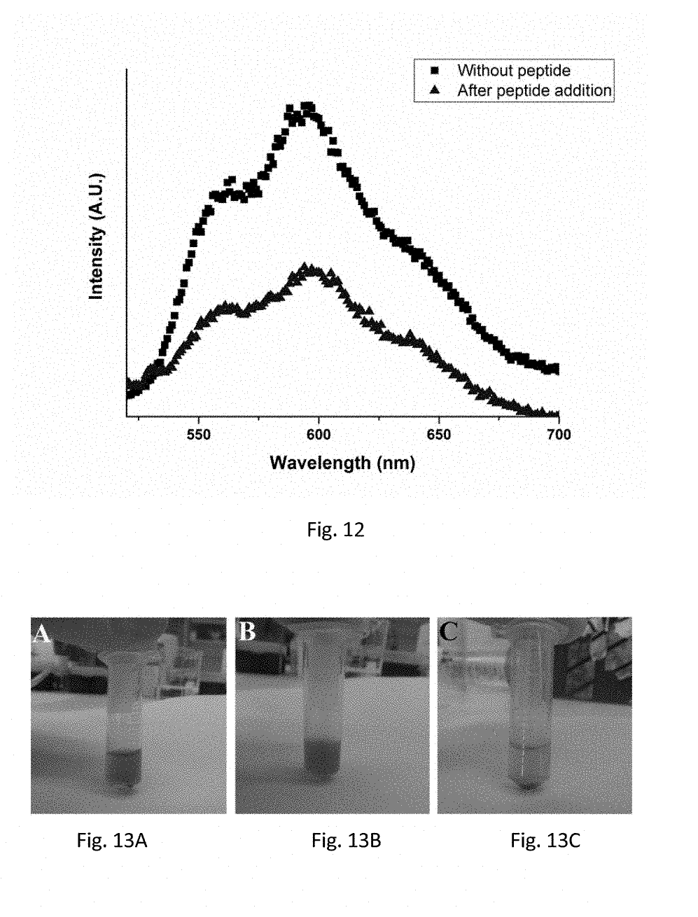

To further investigate the association of the drug with the peptide, we measured the fluorescence spectroscopy. Under the same conditions, the peptide was added to either acidic or basic medium containing the doxorubicin, and the fluorescence intensity at 590 nm before and after the addition was measured. Upon the addition of the peptide, the intensity of the dye markedly decreased (FIG. 12). This can be attributed to the lower amount of free dye in the solution, owing to its encapsulation/adsorption to the peptide assemblies.

To determine whether the peptide assemblies can release the drug, we self-assembled the peptide in the presence of the drug and incubated it overnight. During the incubation, the peptide assemblies precipitated (FIG. 13), and the excess drug and medium could be easily removed. Then, we washed the precipitate and re-dispersed it in PBS (10 mM, pH=7.4). The re-dispersed peptide solutions were transferred to a dialysis device (MWCO 3 kDa), and the fluorescence intensity of the buffer outside the membrane was sampled for 9 days, to detect any traces of the drug. The release results are presented in FIG. 3. In both samples, the one containing the symmetric spheres and the one containing the ‘spikey sphere’ drug traces could be detected and they increased with time. However, the release from the spheres formed in acidic medium was faster, and reached equilibrium after 6 days.

In the case of spheres formed in basic medium, a more controlled release pattern was observed with a linear release. After 9 days, not all of the dye was released. This means that the peptide assemblies can act as an active leaching surface by releasing antibacterial compounds. Based on these results, we attempted to combine the antifouling activity of the peptide with other active compounds against bacteria such as antibiotics and hydrogen peroxide.

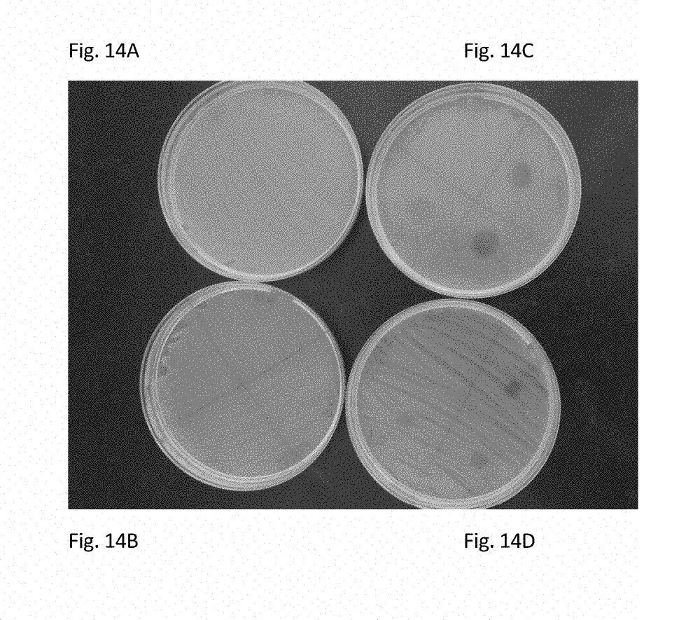

To determine the release of the antibiotic gentamicin from the peptide assemblies, substrates coated with the peptide assemblies entrapping gentamicin were incubated in PBS overnight. Then, a 10 μL drop of the buffer was taken and streaked on agar inoculated with E. coli. After allowing the bacteria to grow, we examined the plates for zones of inhibition. Indeed, clear zones were observed where the buffer drops were applied. However, these zones could not be detected in plates streaked with buffer drops taken at t=0 or drops taken from bare glass slides. The average radius of inhibition from the antibiotic released from the spheres was 0.84±0.07 cm, and from the ‘spikey spheres’ it was 0.94±0.06 cm (FIG. 14). The difference in the size of the zone of inhibition probably resulted from the difference in size: the ‘spikey spheres’ are larger in size than the symmetrical spheres and might entrap a larger amount of antibiotic.

The antifouling and antibacterial activity of peptide assemblies encapsulating Glucose Oxidase (GOx) were also tested. GOx is an enzyme that oxidizes glucose in the presence of oxygen into gluconolactone and hydrogen peroxide. Hydrogen peroxide interrupts with the bacterial cell wall, leading to bacterial death. Thus, it was assumed that incorporating the enzyme would improve the antibacterial activity of the surface. Since the enzyme activity is sensitive to pH, the peptide was assembled only under conditions that resulted in the ‘spikey spheres’, since they are formed under milder pH conditions. Bare glass slides and modified glass slides were incubated in the inoculums of E. coli, at an initial concentration of 10⁵ CFUs/mL, for one hour.

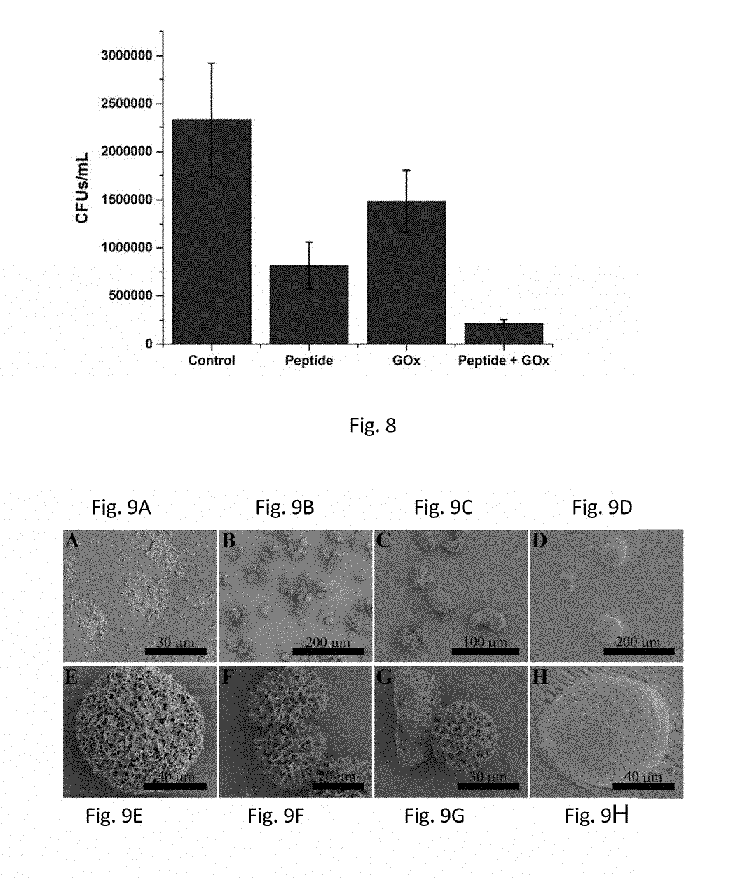

To prove that the peptide assemblies promote the anchoring of GOx to the substrates, we prepared slides on which we drop-casted only GOx. After incubation, the bacteria were removed from the surface by sonication, diluted 10-fold, plated, and counted for CFUs. FIG. 8 summarizes the results.

As observed before, substrates coated with the ‘spikey spheres’ reduced the amount of bacteria by ˜70%. The surfaces that combined the peptide and the GOx exhibited the best activity. The number of bacteria dropped by an order of magnitude, from 2.3×10⁶ CFUs/mL to less than 5×10⁵ CFUs/mL. This proves that combining the peptide activity with the GOx activity improves the overall ability of the surface to resist fouling. It is important to note that although the enzyme was not anchored to the surface, it was found that glass slides coated with GOx alone slightly reduced the amount of bacteria. It is assumed that this is due to some residual enzyme molecules that could form some non-specific interactions with the surface.

### Experimental

### Materials

All chemicals, proteins, and bacteria were purchased from commercially available companies and used as supplied unless otherwise stated. The reported peptide was synthesized by a conventional solution-phase method as described before. Doxorubicin, gentamicin, BSA, and GOx were obtained from Sigma-Aldrich (Jerusalem, Israel). Eschrichia coli (ATCC 25922) was purchased from ATCC (Virginia, USA). Luria broth and tryptic soy broth were obtained from BD difco (New Jersey, USA). Nutrient agar was obtained from Merck (Darmstadt, Germany).

### Stock Solution

To avoid any pre-aggregation, a new fresh stock solution was prepared for each experiment. The fresh stock solution was prepared by dissolving the peptide in pure ethanol (Gadot, Israel) to a concentration of 100 mg/mL.

### Preparation of Porous Spheres

The peptide stock solution was diluted to a final concentration of 1 mg/mL in Tris buffer (10 mM, pH=8.5).

### Preparation of Symmetric Spheres

The peptide stock solution was diluted to a final concentration of 1 mg/mL in 1M HCl solution.

### High Resolution Electron Microscopy (HR-SEM)

A 30 μL drop containing the peptide spheres was drop-casted on a glass cover slip and allowed to dry at room temperature (RT). The peptides on the glass were coated with gold using a Polaron SC7640 sputter coater. SEM images were taken using an extra high-resolution scanning electron microscope, Magellan TM400L, operating at 2 kV.

### Fourier Transform Infrared Spectroscopy (FT-IR)

Peptide stock solution was diluted to a final concentration of 1 mg/mL in duterated media (Tris buffer in D20 or 1M DC1). Then, each peptide solution was deposited on a CaF2 plate and dried under vacuum. Infrared spectra were recorded using a Nicolet 6700 FT-IR spectrometer with a deuterated triglycine sulfate (DTGS) detector (Thermo Fisher Scientific, MA, USA). The measurements were taken using 4 cm⁻¹ resolution and an average of 2000 scans. The absorbance maximal values were determined by the OMNIC analysis program (Nicolet). Each spectrum was deconvoluted.

### Contact Angle Measurements

Contact angle measurements were carried out using a Theta Lite optical tensiometer (Attension, Finland).

### Preparation of Doxorubicin-Loaded Spheres

Doxorubicin-loaded spheres were prepared by diluting a peptide stock solution (100 mg/mL) to a concentration of 1 mg/mL in aqueous medium (either Tris buffer or HCl) containing doxorubicin at a concentration of 0.05 mg/mL.

### Fluorescence Microscopy

A 30 μL drop containing the doxorubicin-loaded sphere solution was drop-casted on a glass cover slip and allowed to dry at RT. A 30 μL drop of doxorubicin in aqueous medium (HCl or tris buffer) was also drop-casted on a glass cover slip as a control. Images were taken using a fluorescence microscope (Carl Zeiss, Axio Vision). Samples were excited at 510 nm.

### Drug Release Study

Doxorubicin-loaded spheres were prepared as described. After the self-assembly process, samples were left to precipitate overnight, the aqueous medium was decanted, and the peptide assemblies were re-dispersed in PBS (10 mM NaCl, pH=7.4, 150 mM). Then, 2 mL of the doxorubicin-loaded peptide were transferred into a dialysis bag (MWCO 3 kDa), and the bag was immersed in 45 mL of PBS, at RT. One mL of the buffer outside the dialysis bag was taken at different time intervals for 9 days, for fluorescence measurements. The volume of the solution was kept constant by adding 1 mL of the original PBS solution after each sampling. The fluorescence measurements were performed at RT using a fluorescence spectrometer (Edinburgh instruments FLS920). The emission spectra were collected from 500 nm to 750 nm, with an excitation wavelength of 480 nm.

### Protein Adsorption

Fifty μL of BSA solution (150 μM in PBS) were applied onto the substrate in a Petri dish. The plate was placed in a humidified incubator at 37° C. for 2 hours. The substrates were then rinsed 3 times with PBS (pH=7.43, 10 mM NaCl, 150 mM), and transferred into test tubes with 1 mL of 2% (w/w) SDS. The samples were shaken for 60 minutes and sonicated for 20 minutes at room temperature to detach the adsorbed proteins. Protein concentrations in the SDS solution were determined using the Non-interfering protein assay (Calbiochem, USA) according to the manufacturer's instructions, using a microplate reader (Synergy 2, BioTek) at 480 nm. All measurements were performed in triplicate and averaged.

### Preparation of Gentamicin-Loaded Spheres

Gentamicin-loaded spheres were prepared by diluting the peptide stock solution (100 mg/mL) to a concentration of 1 mg/mL in aqueous medium (either Tris buffer or HCl) containing gentamicin at a concentration of 0.5 mg/mL.

Preparation of Surfaces Coated with Gentamicin-Loaded Spheres

A 100 μL solution containing gentamicin-loaded spheres was drop-casted on 1×1 cm² glass substrates. Then, the samples were left to dry, dipped in TDW to remove excess drug and peptide, and dried again.

Gentamicin Release from Surfaces

1×1 cm² glass substrates coated with gentamicin-loaded spheres were placed in 3 mL of PBS (10 mM, pH=7.4) and incubated for 24 hours. After incubation, 10 μL were collected from the PBS and placed on an agar plate streaked with E. coli. The plates were incubated at 37° C. overnight. After incubation, the zone of the inhibition diameters were measured, and the amount of antibiotic release was calculated using a calibration chart.

### Preparation of GOx-Loaded Spheres

GOx-loaded spheres were prepared by diluting a peptide stock solution (100 mg/mL) to a concentration of 1 mg/mL in Tris buffer containing GOx at a concentration of 100 μg/mL.

Preparation of Surfaces Coated with GOx-Loaded Spheres

A 150 μL of solution containing GOx-loaded spheres was drop-casted on 1×1 cm² glass substrates. Then, the samples were left to dry, dipped in TDW to remove excess enzyme and peptide, and dried again.

### Bacterial Growth

E. coli (ATCC 25922) were grown in TSB medium at 37° C., for 6 hours, in loosely capped tubes with agitation (120 rpm), to late logarithmic phase. Then, the bacteria were centrifuged and washed 3 times with PBS, re-suspended, and diluted to 10⁵ CFU/mL with TSB.

### Antifouling Activity of the Peptide Assemblies

Three mL of the culture were transferred to each petri dish, and the substrates were placed horizontally in the plate, and incubated in a humidified incubator at 37° C. for either 24 hours or 3 days. One additional mL of TSB was added to each plate after two days to ensure a sufficient supply of nutrients.

After incubation, the substrates were gently rinsed with 3 mL of PBS, and transferred into test tubes with 5 mL of PBS. Then, the test tubes were sonicated for 1 minute to detach bacteria from the substrates, and vortexed for 15 seconds. The number of viable bacteria was determined by plating the samples in 10-fold serial dilutions on LB agar plates.

### Antibacterial Dual Activity of the Surfaces

150 μL of the bacterial culture were gently placed on each substrate, and the substrates were placed horizontally in the plate and incubated in a humidified incubator at 37° C. for 1 hour.

After incubation, the substrates were gently rinsed with 1 mL of PBS, and transferred into test tubes with 3 mL of PBS. Then, the test tubes were sonicated for 1 minute to detach bacteria from the substrates, and vortexed for 15 seconds. The number of viable bacteria was determined by plating the samples in 10-fold serial dilutions on LB agar plates.

1.-61. (canceled)

62. A process for forming particles of an antifouling material, the process comprising contacting an antifouling material, in a non-particulate form, the material having at least one surface binding moiety, at least one antifouling moiety, and optionally at least one amino acid moiety with an aqueous medium under conditions permitting transformation of said material into particles having porosity dependent on the acidity of the aqueous medium.

63. The process according to claim 62, further comprising a step of isolating the particles from the aqueous medium.

64. The process according to claim 62, wherein the medium having a pH between 7 and 10, or between 7 and 9.

65. The process according to claim 62, wherein the medium having a pH between 2 and 5, or between 2 and 4, or between 2 and 3.

66. The process according to claim 62, wherein the particle porosity is characterized by a plurality of pores having pore densities of between about 10 pores/mm² and about 10 pores/100 μm².

67. The process according to claim 66, wherein the pore density is between about 10 pores/mm² and about 10 pores/90 μm², between about 10 pores/mm² and about 10 pores/80 μm², between about 10 pores/mm² and about 10 pores/70 μm², between about 10 pores/mm² and about 10 pores/60 μm², between about 10 pores/mm² and about 10 pores/50 μm², between about 10 pores/mm² and about 10 pores/40 μm², between about 10 pores/mm² and about 10 pores/30 μm², between about 10 pores/mm² and about 10 pores/20 μm², between about 10 pores/mm² and about 10 pores/10 μm², between about 10 pores/mm² and about 10 pores/5 μm², between about 10 pores/mm² and about 10 pores/2 μm² or between about 10 pores/mm² and about 10 pores/1 μm².

68. The process according to claim 66, wherein the pore density is between about 20 pores/mm² and about 10 pores/100 μm², between about 30 pores/mm² and about 10 pores/100 μm², between about 40 pores/mm² and about 10 pores/100 μm², between about 50 pores/mm² and about 10 pores/100 μm², between about 60 pores/mm² and about 10 pores/100 μm², between about 70 pores/mm² and about 10 pores/100 μm², between about 80 pores/mm² and about 10 pores/100 μm², between about 90 pores/mm² and about 10 pores/100 μm², between about 100 pores/mm² and about 10 pores/100 μm², between about 110 pores/mm² and about 10 pores/100 μm², between about 120 pores/mm² and about 10 pores/100 μm², between about 130 pores/mm² and about 10 pores/100 μm², between about 140 pores/mm² and about 10 pores/100 μm², between about 150 pores/mm² and about 10 pores/100 μm², between about 160 pores/mm² and about 10 pores/100 μm², between about 170 pores/mm² and about 10 pores/100 μm², between about 180 pores/mm² and about 10 pores/100 μm², between about 190 pores/mm² and about 10 pores/100 μm² or between about 200 pores/mm² and about 10 pores/100 μm².

69. The process according to claim 62, wherein the average pore diameter is between 2 and 50, between 2 and 40, between 2 and 35, between 2 and 30, between 2 and 25, between 2 and 20, between 2 and 15, between 2 and 10, between 10 and 100, between 10 and 90, between 10 and 80, between 10 and 70, between 10 and 60, between 10 and 50, between 10 and 40, between 10 and 30, or between 10 and 20 micrometer.

70. Particles of a material having at least one surface binding moiety, at least one antifouling moiety, and optionally at least one amino acid moiety, the particles being porous.

71. The particles according to claim 70, entrapping, associating, encaging, containing or holding one or more active or non-active materials in cavities or pores present in the particles or on outer surfaces of the particles.

72. The particles according to claim 70, wherein the material is of the general Formula I:
J-L-X-B
wherein
J is a surface binding moiety,
X is an antifouling moiety,
L is a covalent bond or a linker moiety linking J and X,
B may be absent or an amino acid moiety, and
each of “-” represents a bond.

73. The particles according to claim 72, wherein, where L is present, it is bonded to each of J and X via covalent bonds or non-hydrolysable bonds.

74. The particles according to claim 72, wherein, where L is absent, J and X are bonded to each other via a covalent bond or non-hydrolysable bond.

75. The particles according to claim 72, wherein the surface binding moiety is selected from the group consisting of one or more 3,4-dihydroxy-L-phenylalanin (DOPA), DOPA containing moiety, dopamine and trihydroxyphenylalanine.

76. The particles according to claim 72, wherein the antifouling moiety is a fluorine (—F) atom or a group comprising at least one fluorine atom.

77. The particles according to claim 76, wherein the antifouling moiety is a fluorinated carbon group.

78. The particles according to claim 72, wherein the material is selected from the group consisting of: J-X-B, J-B-X, X-B-J, X-J-B, B-X-J and B-J-X; wherein each of B, J, X and “-” are selected as defined in claim 72.

79. The particles according to claim 78, wherein B is at least one amino acid sequence promoting adherence of cells, optionally selected from the group consisting of RGD (Arg-Gly-Asp); KQAGDV; YIGSR; REDV; IKVAV; RNIAEIIKDI; KHIFSDDSSE; VPGIG; FHRRIKA; KRSR; NSPVNSKIPKACCVPTELSAI; APGL; VRN; and AAAAAAAAA.

80. The particles according to claim 72, wherein the material is selected from the group consisting of:

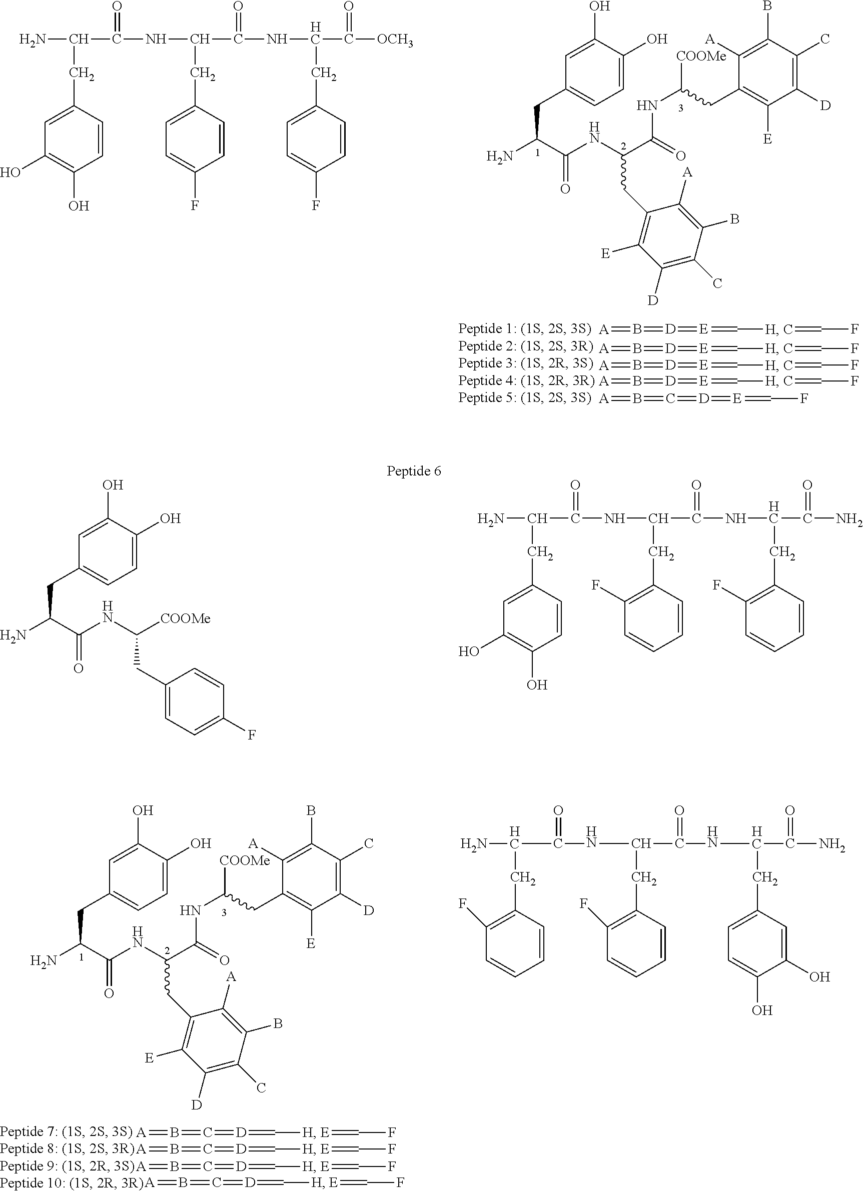

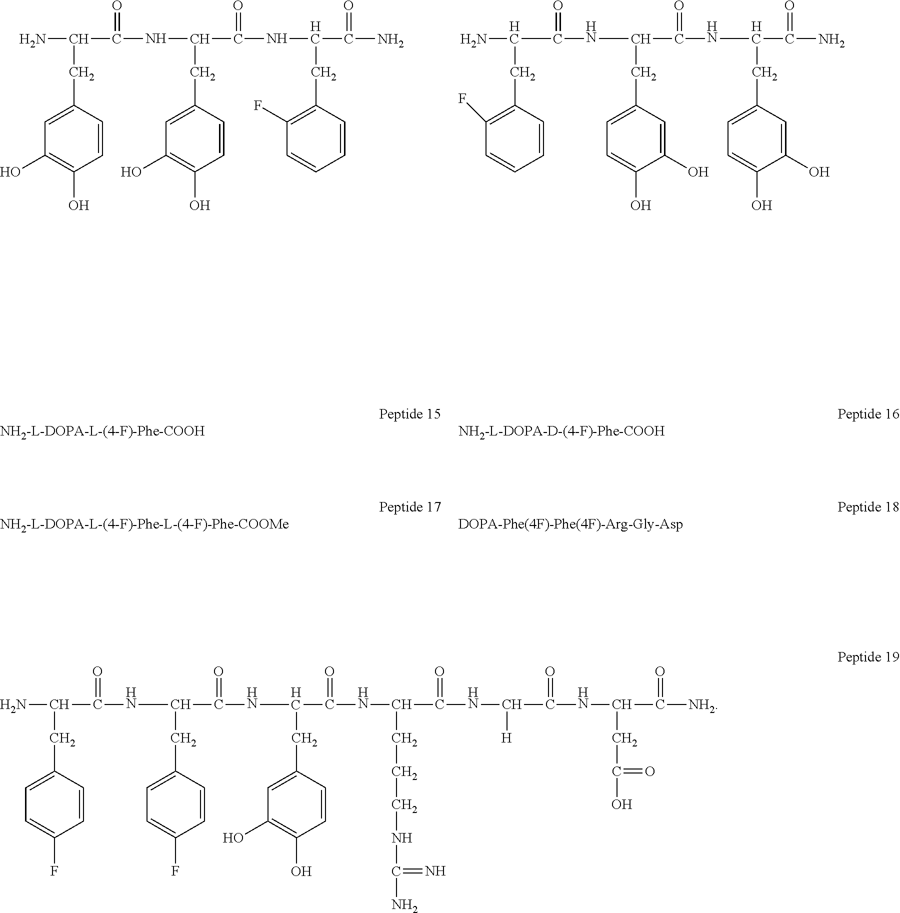 [SMILES: NC(=O)C(Cc1ccccc1F)NC(=O)C(Cc1ccc(O)c(O)c1)NC(=O)C(N)Cc1ccc(O)c(O)c1 ; NC(=O)C(Cc1ccc(O)c(O)c1)NC(=O)C(Cc1ccc(O)c(O)c1)NC(=O)C(N)Cc1ccccc1F ; N=C(N)NCCCC(NC(=O)C(Cc1ccc(O)c(O)c1)NC(=O)C(Cc1ccc(F)cc1)NC(=O)C(N)Cc1ccc(F)cc1)C(=O)NCC(=O)NC(CC(=O)O)C(N)=O]

81. A medical device or implant having at least a surface region thereof coated with a plurality of particles according to claim 70.
